(19) United States
(12) Patent Application Publication
(10) Pub. No.: US 2026/0022370 A1
(43) Pub. Date: Jan. 22, 2026

(54) SYNTHETIC ENHANCERS AND PROMOTERS BASED ON CONCATENATED PALINDROMIC SUBSEQUENCES

(71) Applicant: THE GOVERNING COUNCIL OF THE UNIVERSITY OF TORONTO, Toronto (CA)

(72) Inventors: Kien TRUONG, Thornhill (CA); Polina GOVORKOVA, Burlington (CA)

(73) Assignee: THE GOVERNING COUNCIL OF THE UNIVERSITY OF TORONTO, Toronto, ON (CA)

(21) Appl. No.: 18/838,341

(22) PCT Filed: Feb. 18, 2023
(86) PCT No.: PCT/CA2023/050215

§ 371 Date: Aug. 14, 2024

Related U.S. Application Data
(60) Provisional application No. 63/268,234, filed on Feb. 18, 2022.

(51) Int. Cl. C12N 15/10; G16B 25/20; G16B 40/20

(52) U.S. Cl. CPC C12N 15/1082; C12N 15/1089; G16B 25/20; G16B 40/20

(57) ABSTRACT

The present disclosure relates to a method of constructing a synthetic enhancer comprising: identifying probable palindromic subsequences in a promoter of interest; selecting and extracting highly palindromic subsequences from amongst the probable palindromic subsequences; and concatenating some or all of the extracted highly palindromic subsequences to produce a synthetic enhancer. Identifying the probable palindromic subsequence includes defining a candidate subsequence in the promoter of interest; generating a complement or reverse complement of the candidate subsequence; comparing the candidate subsequence with its complement or reverse complement to identify the number of mismatches; and identifying the candidate subsequence as a probable palindromic subsequence if the number of mismatches is the same or lower than a mismatch threshold corresponding to the number of mismatches expected from comparable randomly generated sequences. The method can be applied to create a synthetic enhancer for any promoter of interest.

### CROSS-REFERENCE TO RELATED APPLICATIONS

This application is the National Phase application of PCT International Patent Application No. PCT/CA2023/050215, filed on Feb. 18, 2023 and titled “SYNTHETIC ENHANCERS AND PROMOTERS BASED ON CONCATENATED PALINDROMIC SUBSEQUENCES,’ which claims benefit of priority under 35 U.S.C. 119 to U.S. Provisional Patent Application Ser. No. 63/268,234, filed Feb. 18, 2022, and titled “SYNTHETIC ENHANCERS AND PROMOTERS BASED ON CONCATENATED PALINDROMIC SUBSEQUENCES,” the contents of each application are herein incorporated by reference in its entirety.

### SEQUENCE LISTING

The instant application contains a Sequence Listing which has been submitted in XML file format via Patent Center and is hereby incorporated by reference in its entirety. Said XML file copy, created on Aug. 1, 2025, is named Sequence_Listing_17978-275.xml, and is 73,168,722 bytes in size. The Sequence Listing is incorporated by reference in its entirety.

### TECHNICAL FIELD

This disclosure generally relates to synthetic enhancers and promoters for driving transcription in host cells. More specifically, this disclosure relates to a method of designing synthetic promoters using concatenated palindromic subsequences.

### BACKGROUND

To express transgenes in specific cell types and states, promoters for endogenous genes are commonly created by truncating the sequence upstream of the transcriptional start site until the promoter is no longer functional to determine a minimum region of nucleotides required for a functional promoter. This method of designing truncated promoters often results in a promoter sequence that is longer than necessary. Typically, shorter promoter sequences are desired as gene delivery efficiency decreases with the increasing length of genetic material.

In cases where expression is required for specific tissues, the promoters for endogenous genes that are expressed in relatively greater concentration than other tissues, such as the synapsin-1 promoter in neurons, are often used. While the consensus binding sequences for some transcription factors have been experimentally determined, there remain many whose consensus binding sequences are unknown. Thus, designing a minimal synthetic enhancer region for these endogenous promoters is not always possible. As a result, the design of these promoters typically begins with the synthesis of a subsequence of the promoter between ˜1000 nucleotides upstream and ˜50 nucleotides downstream of the transcription start site (TSS). Then, the upstream section is truncated at the 5′ end until the promoter no longer functions as desired. For example, to isolate the active regions of the human synapsin-1 promoter, 5′ end truncations were performed until a minimal region 422 nucleotides upstream of the TSS was identified that retained strong expression in PC12 neuronal cells compared to non-neuronal cells. While this 5′ end truncation strategy may work for some promoters, many 5′ end truncated sequences need to be synthesized before finding the optimal one. Moreover, even after the optimal truncation is found, it may still contain subsequences that do not contribute to the promoter functionality.

While many methods have been developed to efficiently find biological palindromes in sequences, it is difficult to determine which palindromes are truly significant. For example, short palindromes (i.e., six nucleotides) may bind transcription factors, but they occur too frequently to effectively distinguish between transcriptional function and random occurrence.

### SUMMARY

According to one aspect, there is provided a method of constructing a synthetic enhancer, the method comprising: identifying probable palindromic subsequences in a promoter of interest; selecting and extracting highly palindromic subsequences from amongst the probable palindromic subsequences, the highly palindromic subsequences being those having a palindromic density above a palindromic density threshold; and concatenating some or all of the extracted highly palindromic subsequences to produce a synthetic enhancer having an overall length that is less than that of a contiguous segment of the promoter of interest that comprises all the concatenated highly palindromic subsequences.

In some embodiments, identifying probable palindromic subsequences comprises: defining a candidate subsequence of a predetermined length in the promoter of interest; generating a complement or reverse complement of the candidate subsequence; comparing the candidate subsequence with its complement or reverse complement to identify the number of mismatches; and identifying the candidate subsequence as a probable palindromic subsequence if the number of mismatches is the same or lower than a mismatch threshold corresponding to the number of mismatches expected from comparable randomly generated sequences.

In some embodiments, selecting highly palindromic subsequences based on palindromic density comprises determining a palindromic nucleotide score for each individual nucleotide in the probable palindromic subsequence, the palindromic nucleotide score correlating with the number of probable palindromic subsequences of different lengths and different subsequence frames in which the nucleotide participates, and optionally plotting a palindromic density graph of palindromic nucleotide score as a function of nucleotide position within the promoter of interest. In some embodiments, selecting highly palindromic subsequences based on palindromic density further comprises determining an overall palindromic density sequence score for each probable palindromic subsequence, the overall palindromic density sequence score correlating with the palindromic nucleotide scores for all or substantially all individual nucleotides in the probable palindromic subsequence. In some embodiments, the palindromic density threshold is based on the expected palindromic densities of comparable randomly generated sequences.

In some embodiments, the method further comprises synthesizing a polynucleotide comprising the synthetic enhancer. In some embodiments, the synthetic enhancer is fused to a core promoter, or to a core promoter operably fused to a polynucleotide sequence to be transcribed. In some embodiments, the synthetic enhancer is heterologous with respect to the core promoter and/or with respect to the polynucleotide sequence to be transcribed.

According to another aspect, there is provided a method of constructing a synthetic promoter, the method comprising: providing the synthetic enhancer produced by or as defined herein; and operably linking the synthetic enhancer to a core promoter as defined herein. According to another aspect, there is provided a synthetic promoter suitable for driving transcription of a DNA sequence of interest, wherein the synthetic promoter is as defined herein, or is constructed by the method as defined herein. According to another aspect, there is provided an expression cassette or vector comprising the synthetic enhancer produced by or as defined herein operably linked to a core promoter as defined herein.

In some embodiments, the synthetic promoter defined herein, or the expression cassette or vector defined herein, is for use in gene therapy. In some embodiments, the synthetic promoter defined herein, or the expression cassette or vector defined herein, is for use in genome editing, wherein the synthetic promoter drives expression of an endonuclease (e.g., an RNA-guided endonuclease) and/or a guide RNA.

According to another aspect, there is provided a computer-implemented process for constructing a synthetic enhancer, the process comprising: (a) inputting or receiving a nucleotide sequence of a promoter of interest; (b) identifying probable palindromic subsequences in the nucleotide sequence of the promoter of interest; (c) selecting and extracting highly palindromic subsequences from amongst the probable palindromic subsequences, the highly palindromic subsequences being those having a palindromic density above a palindromic density threshold; and (d) concatenating some or all of the extracted highly palindromic subsequences to produce a synthetic enhancer having an overall length that is less than that of a contiguous segment of the promoter of interest that comprises all the concatenated highly palindromic subsequences. In some embodiments, the computer-implemented process is a cloud-based computer-implemented process. In some embodiments, the computer is configured to implement the method as defined herein.

According to another aspect, there is provided a non-transitory computer-readable medium storing processor-executable instructions, the instructions when executed by a processor cause the processor to perform the method as defined herein, and optionally outputting sequence information to a user.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one,” but it is also consistent with the meaning of “one or more”, “at least one”, and “one or more than one”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

### BRIEF DESCRIPTION OF THE DRAWINGS

FIG. 1A shows a representation of a direct binding of oligomeric transcription factors to a palindromic sequence (striped rectangle); FIG. 1B shows a representation of an indirect binding of oligomeric transcription factors to a palindromic sequence (striped rectangle); and FIG. 1C shows an example calculation of a mismatch indicator function M(s, i) and the summation of mismatches N(s) in a given sequence s, in which the 8 nucleotide long sequence s is shown in bold text, a complement and reverse of the sequence s (C(a)) is shown in unbold text, and the mismatches are indicated with downward-pointing arrows.

FIG. 2 shows a flow chart of an exemplary method of identifying highly palindromic subsequences.

FIGS. 3A to 3D show bar graphs representing the probability of the summation of mismatches N(s) in random sequences s having lengths of 6 nucleotides (FIG. 3A); 10 nucleotides (FIG. 3B); 20 nucleotides (FIG. 3C); and 30 nucleotides (FIG. 3D). An exemplary mismatch threshold of the number of mismatches expected from the top 2% to 3% of randomly generated sequences of the same length is shown as a broken line with probable palindromes being indicated with arrow.

FIGS. 4A to 4D show exemplary palindromic density graphs representing the palindromic score S(s, i) of each nucleotide in an exemplary sequence having 18 nucleotides and exhibiting long palindromes (FIG. 4A) or overlapping palindromes (FIG. 4C). The corresponding palindromic line graphs of the palindromic score S(s, i) at each nucleotide index i are shown for long palindromes (FIG. 4B) exhibiting a sharp peak and overlapping palindromes exhibiting a flatter peak (FIG. 4D).

FIGS. 5A to 5F show line graphs representing the palindromic score S(s, i) for each nucleotide at nucleotide index i for three random 1101 nucleotide sequences marked as yellow, red, and blue (FIG. 5A); a CMV promoter (FIG. 5B); a human insulin promoter (FIG. 5C); a human desmin promoter (FIG. 5D); a human synapsin-1 promoter (FIG. 5E); and a truncated human synapsin-1 promoter (InvivoGen; FIG. 5F). The nucleotide index i of the transcription start site (TSS) is represented by a vertical broken line.

FIGS. 6A and 6B show line graphs representing: the probability distribution of the palindromic scores S(s, i) found in randomly generated sequences (yellow), Mus musculus version mm 10 genome promoters (black), and Homo sapien hg38 genome promoters (red) (FIG. 6A); and the probability distribution of the average palindromic scores A(s) for each of the randomly generated sequences (yellow), each Mus musculus version mm 10 genome promoter (black), and each Homo sapien hg38 genome promoter (red) (FIG. 6B).

FIGS. 7A to 7D shows line graphs representing the palindromic score S(s, i) for each nucleotide at nucleotide index i for orthologous promoters of the synapsin-1 promoter in Mus musculus (FIG. 7A); Sus domesticus (pig) (FIG. 7B); Rattus norvegicus domestica (rat) (FIG. 7C); and Drosophila melanogaster (fly) (FIG. 7D). The nucleotide index i of the TSS is represented by a vertical broken line.

FIG. 8A shows a line graph representing the probability distribution of the palindromic scores S(s, i) of 1,003,000,000 fully scored nucleotides from 1,000,000 randomly generated sequences. The exemplary enhancement threshold of the top 25% of palindromic scores S(s, i), corresponding to a palindromic score S(s, i) of at least 40, is shown with a vertical broken line. FIG. 8B shows a flow chart of an exemplary method of designing a synthetic promoter. FIG. 8C shows a line graph representing the palindromic score S(s, i) at each nucleotide index i for the cytomegalovirus immediate-early (CMV) promoter. The highly palindromic subsequences comprising nucleotides with a palindromic score S(s, i) above the enhancement threshold (i.e., at least 40) are indicated by a broken rectangle, with the remaining promoter nucleotides that were not deemed to be highly palindromic falling outside of the broken rectangle. The TSS is represented by a vertical solid line. FIG. 8D shows the nucleotide sequence of the synthetic CMV promoter (SEQ ID NO: 54696) developed using the method described herein. The nucleotides of a CREB site in the forward direction are highlighted in black and in the reverse direction are underlined. The bold nucleotides indicate an AP-1 site. The minimal CMV core promoter is shown in italics. FIG. 8E a line graph representing the palindromic score S(s, i) at each nucleotide index i for the mouse synapsin-1 promoter. The highly palindromic subsequences are indicated by a broken rectangle, with the remaining promoter nucleotides that were not deemed to be highly palindromic falling outside of the broken rectangle. The TSS is represented by a solid vertical line. FIG. 8F shows the nucleotide sequence of the synthetic mouse synapsin-1 promoter (SEQ ID NO: 54698) developed using the method described herein. The nucleotides of a NRSE/RE-1 site are highlighted in black, and the minimal CMV core promoter is shown in italics.

FIG. 9A shows a schematic of Venus yellow fluorescent protein constructs regulated by synthetic promoters derived from the CMV promoter (PCMVp, SEQ ID NO: 54696) and the mouse synapsin-1 promoter (PmSyn-1, SEQ ID NO: 54698). FIG. 9B shows a representation of the translocation of the fluorescent reporter to the plasma membrane by post-translational lipid modification. FIG. 9C shows a 10× magnification representative fluorescence image of HEK293 cells transiently transfected with the PCMVp construct. FIG. 9D shows a 40× magnification representative fluorescence image of HEK293 cells transiently transfected with the PCMVp construct. FIG. 9E shows a 10× magnification representative fluorescence image of N2A cells transiently transfected with the PmSyn1 construct. FIG. 9F shows a 40× magnification representative fluorescence image of N2A cells transiently transfected with the PmSyn1 construct. FIG. 9G shows a bar graph representing the percentage of Venus positive cells after transfection with a minimal core CMV promoter (core, SEQ ID NO: 1), CMVp (full promoter, SEQ ID NO: 54700), and PCMVp (short synthetic promoter, SEQ ID NO: 54696). FIG. 9H shows a bar graph representing the percentage of Venus positive cells after transfection with a minimal core CMV promoter (core, SEQ ID NO: 1), mSyn1p (full promoter, SEQ ID NO: 54699), and PmSyn4p (short synthetic promoter, SEQ ID NO: 54698). FIG. 9I shows a bar graph representing the percentage of Venus positive cells in N2A cells, Hela cells, MDCK cells, CHO cells, and 3T3 cells after transfection with PmSyn1p (SEQ ID NO: 54698).

FIG. 10A to 10C shows bar graphs representing the normalized fluorescence intensity (f/f₀) of HEK293 cells transfected with CMVp (full promoter, SEQ ID NO: 54700) and PCMVp (short synthetic promoter, SEQ ID NO: 54696) (FIG. 10A); the normalized fluorescence intensity (f/f₀) of N2A cells transfected with mSyn1p (full promoter, SEQ ID NO: 54699) and PmSyn1p (short synthetic promoter, SEQ ID NO: 54698) (FIG. 10B); and the percentage of Venus positive cells in N2A cells, Hela cells, MDCK cells, CHO cells, and 3T3 cells after transfection with CMVp (full promoter, SEQ ID NO: 54700) (FIG. 10C).

FIG. 11 shows a bar graph representing the percentage of Venus positive cells in HEK293 cells after transfection with the following short synthetic promoters: PCMVp (SEQ ID NO: 54696), PhCALRp (SEQ ID NO: 54701), PhEF1p (SEQ ID NO: 54702), PhHSP70p (SEQ ID NO: 54703), PhLDHAp (SEQ ID NO: 54704), PhNPM1p (SEQ ID NO: 54705), PhPKMp (SEQ ID NO: 54706), PhRACK1p (SEQ ID NO: 54707), PhTUBA1p (SEQ ID NO: 54708), PhUBBp (SEQ ID NO: 54709), and PhUBCp (SEQ ID NO: 54710).

FIG. 12 shows a bar graph representing the probability distribution of the length of synthetic enhancers having nucleotides sequences as shown in SEQ ID NOs: 2 to 54695 that were designed for all promoters in the Homo sapiens and Mus musculus genomes using the methods described herein.

### SEQUENCE LISTING

The present application is being filed along with a Sequence Listing in electronic format that was created on February 18, 2023. The information in electronic format of the Sequence Listing is incorporated herein by reference in its entirety.

The nucleotide sequence of the CMV core promoter (core) is shown in SEQ ID NO: 1. The nucleotide sequences of synthetic enhancers extracted from promoters in the Homo sapien genome are shown in SEQ ID NOs: 2 to 29597. The nucleotide sequences of synthetic enhancers extracted from promoters in the Mus musculus genome are shown in SEQ ID NOs: 29598 to 54695. The organism name indicated for each of the synthetic constructs of SEQ ID NOs: 2 to 54695 includes both the gene name and organism from which each of the synthetic enhancers and/or promoters were derived. In some instances, the gene name is followed by an underscore and a number, which refers to different transcriptional start sites that have been identified for that gene starting from the most upstream transcriptional start site.

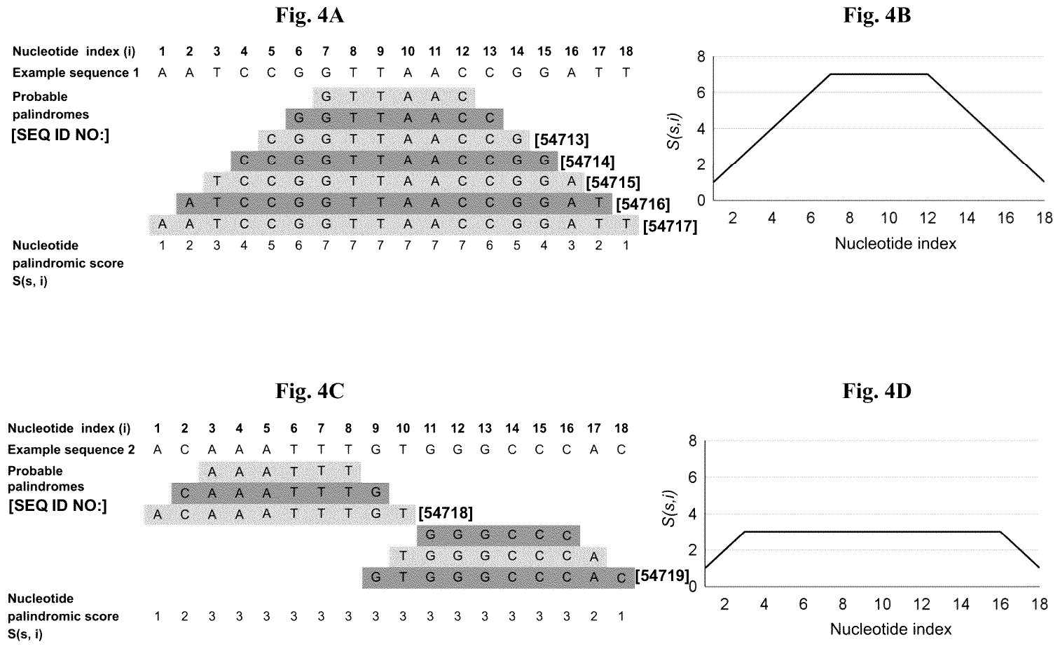

A synthetic CMV promoter (PCMVp) comprising a synthetic CMV enhancer and the minimal CMV core promoter is shown in SEQ ID NO: 54696. The nucleotide sequence of the synthetic CMV enhancer is shown in SEQ ID NO: 54697 and the nucleotide sequence of the full CMV promoter is shown in SEQ ID NO: 54700. The nucleotide sequence of a full mouse synapsin-1 promoter (mSyn1p) used as a control is shown in SEQ ID NO: 54699 and the nucleotide sequence of a synthetic mouse synapsin-1 promoter (PmSyn1p) is shown in SEQ ID NO: 54698. The nucleotide sequence of synthetic promoters extracted from full promoters of the following Homo sapiens genes: CALR, EEF1A1, HSP70, LDHA, NPM1, PKM, RACK1, TUBA1, UBB, and UBC are shown in SEQ ID NOs: 54701 to 54710, respectively. The nucleotide sequence of a serum response factor (SRF) is shown in SEQ ID NO: 54711. The amino acid sequence of the 12 N-terminal amino acids of a Lyn kinase is shown in SEQ ID NO: 54712. The synthetic constructs showing example probable palindromes in FIG. 4A are identified in SEQ ID NOs: 54713 to 54717 and the synthetic constructs showing example probable palindromes in FIG. 4C are identified in SEQ ID NOs: 54718 and 54719.

### DETAILED DESCRIPTION

A method of designing shortened synthetic enhancers and promoters for gene expression, and the synthetic enhancers and promoters created by such method, are described herein. In general, the synthetic enhancers described herein are constructed by identifying highly palindromic nucleotides within the sequence of a promoter of interest using a palindromic density metric. Highly palindromic subsequences are then concatenated to create a synthetic enhancer that is significantly shorter in overall length as compared to corresponding sequences comprised in the original promoter of interest. Strikingly, in some embodiments, the shortened synthetic enhancers described herein retain promoter-enhancing activity and/or tissue-specificity comparable to that of their parent full-length promoters.

In a first aspect, described herein is a method of constructing a synthetic enhancer (or a candidate synthetic enhancer). The method generally comprises: identifying probable palindromic subsequences in a promoter of interest; selecting and extracting highly palindromic subsequences from amongst the probable palindromic subsequences (e.g., highly palindromic subsequences having a palindromic density above a given palindromic density threshold); and concatenating some or all of the extracted highly palindromic subsequences to produce a synthetic enhancer having an overall length that is less than that of a contiguous segment of the promoter of interest that comprises all the concatenated highly palindromic subsequences. As used herein, the term “synthetic” in the expressions “synthetic enhancer” and “synthetic promoter” refer to sequences that are not found in a genome of naturally-occurring or non-genetically modified organism. As used herein, the terms “enhancer” or “synthetic enhancer” refer to sequences having promoter-enhancing activity—i.e., they can activate, improve, or functionally modify (e.g., control tissue-specific expression) a core promoter's transcriptional activity when fused thereto.

### Identifying Probable Palindromic Subsequences

In some embodiments, identifying probable palindromic subsequences may comprise: defining a candidate subsequence of a predetermined length in the promoter of interest; generating a complement or reverse complement of the candidate subsequence; comparing the candidate subsequence with its complement or reverse complement to identify the number of mismatches; and identifying the candidate subsequence as a probable palindromic subsequence if the number of mismatches is the same or lower than a mismatch threshold corresponding to the number of mismatches expected from comparable randomly generated sequences.

In some embodiments, the candidate subsequence's length may be set at a minimal length of at least 4, 5, 6, 7, 8, 9, or 10 nucleotides, and/or at a maximal length of up to 30, 35, 40, 45, 50, 55, 60, 65, 70, 75, 80, 85, 90, 95, 100, 125, or 150 nucleotides. In some embodiments, the candidate subsequence may be compared with its reverse complement by performing a sequence alignment to identify the number of mismatches. In some embodiments, the mismatch threshold may be selected or may correspond to the number of mismatches expected from the most palindromic randomly generated sequences of the same or similar length as the candidate subsequence. In some embodiments, the mismatch threshold may be selected as the number of mismatches expected within a given percentile (e.g., 75^{th}, 80^{th}, 85^{th}, 90^{th}, 95^{th}, 96^{th}, 97^{th}, 98^{th} or 99^{th} percentile) of randomly generated sequences of the same or similar length as the candidate subsequence.

In some embodiments, comparing the number of mismatches between the candidate subsequence and its reverse complement may be determined using the mismatch indicator function M(s, i):

$$M ( s , i ) = \{ \begin{matrix}1 , if {s}_{i} \neq C ( {s}_{L ( s ) - i + 1} ) \\ 0 , otherwise\end{matrix} ;$$

where s is a candidate subsequence of the promoter of interest, i is a nucleotide index, L(s) is a length of the subsequence s, and C(s_{L(s)-i+1}) is the DNA complement of the nucleotide identified by s_{L(s)-i+1}. For example, when the nucleotide in the subsequence s at the nucleotide index i is adenine, the DNA complement of the nucleotide (C(s_{L(s)-i+1})) would be thymine. Similarly, when the nucleotide is thymine, guanine, or cytosine, the DNA complement is adenine, cytosine, or guanine, respectively.

In some embodiments, comparing the number of mismatches between the candidate subsequence and its reverse complement may further comprise performing a summation of the mismatches N(s):

$$N ( s ) = {\sum }_{i = 1}^{i = L ( s )} M ( s , i ) .$$

In some embodiments, probable palindromic subsequences may be determined by calculating a probable palindrome indicator function P(s):

$$P ( s ) = \{ \begin{matrix}1 , if N ( s ) \leq Cutoff ( L ( s ) ) \\ 0 , otherwise\end{matrix} ;$$

where Cutoff(p) is a mismatch threshold corresponding to the number of allowed mismatches for a sequence of length p.

### Selecting and Extracting Highly Palindromic Subsequences

In some embodiments, selecting highly palindromic subsequences based on palindromic density may comprise determining a palindromic nucleotide score for each individual nucleotide in the probable palindromic subsequence, the palindromic nucleotide score correlating with the number of probable palindromic subsequences of different lengths and different subsequence frames in which the nucleotide participates. In some embodiments, a palindromic density graph of palindromic nucleotide score as a function of nucleotide position within the promoter of interest may be plotted.

In some embodiments, selecting highly palindromic subsequences based on palindromic density may further comprise determining an overall palindromic density sequence score for each probable palindromic subsequence, wherein the overall palindromic density sequence score correlates with the palindromic nucleotide scores for all (or substantially all) individual nucleotides in the probable palindromic subsequence.

In some embodiments, the palindromic density threshold may be set based on the expected palindromic densities of comparable randomly generated sequences. For example, the palindromic density threshold may be set to be within a 60^{th}, 65^{th}, 70^{th}, 75^{th}, 80^{th}, 85^{th}, 90^{th}, or 95^{th} percentile of the expected palindromic densities of comparable randomly generated sequences.

In some embodiments, the palindromic nucleotide score S(s, i) may be determined by:

$$S ( s , i ) = {\sum }_{p = y}^{p = x} {\sum }_{w = max ( i - p + 1 , 1 )}^{w = min ( i , L ( s ) - p + 1 )} P ( {s}_{w … ( w + p - 1 )} )$$

wherein p is a palindrome length of each probable palindromic subsequence, and the palindrome length has a maximum number of nucleotides equal to x, and a minimum number of nucleotides equal to y. In some embodiments, x may be 30, 35, 40, 45, 50, 55, 60, 65, 70, 75, 80, 85, 90, 95, 100, 125, or 150 nucleotides. In some embodiments, y may be 4, 5, 6, 7, 8, 9, or 10 nucleotides. In some embodiments, the length of the sequence (L(s)) of the promoter of interest may be less than 1 000 000, 500 000, 250 000, 200 000, 150 000, 100 000, 50 000, 25 000, 20 000, 15 000, 10 000, 7500, 5000, 4000, 3000, 2000, 1500, or 1101 nucleotides.

In some embodiments, the overall palindromic density sequence score may be calculated based on the average of the palindromic nucleotide scores of all individual nucleotides in the probable palindromic subsequence according to the function:

$$A ( s ) = \frac{1}{L ( s )} \cdot {\sum }_{i = 1}^{i = L ( s )} S ( s , i )$$

where i is the nucleotide index.

### Concatenation of Highly Palindromic Subsequences

In some embodiments, the method described herein comprise concatenating some or all of the extracted highly palindromic subsequences to produce a synthetic enhancer having an overall length that is less than that of a contiguous segment of the promoter of interest that comprises all the concatenated highly palindromic subsequences. Conversely, methods described herein may comprise removing intervening genomic sequences between highly palindromic subsequences to shorten the overall length of a synthetic enhancer and/or synthetic promoter described herein.

As used herein, the terms “concatenating”, “concatenation” and “concatenated”, and the like, refer to the joining or fusing together of highly palindromic subsequences described herein such that the concatenated sequences have promoter-enhancing activity. For greater clarity, the concatenations described herein are not limited to maintaining the same 5′ to 3′ order in which the highly palindromic subsequences are found in their parental promoter sequences. As there may be great variability in the length of intervening non-highly palindromic genomic sequences, individual highly palindromic subsequences may be viewed as being modular in nature.

In some embodiments, extracted highly palindromic subsequences may be concatenated with one or more intervening synthetic linker sequences therebetween, wherein at least one of synthetic linker synthetic comprises a palindromic subsequence, a non-palindromic subsequence, or binding site (e.g., a restriction site or landing sites, such as an integrase, recombinase, or transposase landing site).

In some embodiments, the synthetic linker sequences may be between 1 and 50, preferably between 1 and 20 nucleotides in length. It is understood that using linkers longer than necessary may undesirably lengthen the overall length of the synthetic enhancer and/or promoter comprising same. Thus, in some embodiments, the extracted highly palindromic subsequences are concatenated without intervening synthetic linker sequences therebetween.

### Promoters of Interest

In some embodiments, promoters of interest described herein may have a length of less than 1 000 000, 500 000, 250 000, 200 000, 150 000, 100 000, 50 000, 25 000, 20 000, 15 000, 10 000, 7500, 5000, 4000, 3000, 2000, 1500, 1250, or 1000 nucleotides. In some embodiments, promoters of interest described herein may comprise between 200 and 5000 nucleotides upstream of a transcription start site of the promoter of interest. In some embodiments, promoters of interest described herein may comprise 0 to 200, 0 to 150, 0 to 100, or 20 to 100 nucleotides downstream of the transcription start site promoter of interest. In some embodiments, promoters of interest described herein may comprise less than 1000 nucleotides upstream of the transcription start site of the promoter of interest.

In some embodiments, promoters of interest described herein may be from a constitutive promoter, an inducible promoter, and/or a tissue-specific promoter.

In some embodiments, promoters of interest described herein may comprise a promoter from a mammalian genome, such as a Homo sapiens genome (e.g., hg38) or a Mus musculus genome (e.g., mm10).

### Synthesis of Synthetic Enhancers and Synthetic Promoters

In some embodiments, methods described herein may comprise synthesizing a polynucleotide comprising a synthetic enhancer as defined herein. In some embodiments, the synthetic enhancer may be fused to a core promoter, or to a core promoter operably fused to a polynucleotide sequence to be transcribed in RNA (e.g., mRNA or non-coding RNA).

In some embodiments, the synthetic enhancer may be heterologous with respect to the core promoter and/or with respect to the polynucleotide sequence to be transcribed. In some embodiments, the core promoter may be from a constitutive promoter, an inducible promoter, and/or a tissue-specific promoter. In some embodiments, the core promoter may be a minimal CMV promoter.

In some embodiments, described herein is a method of constructing a synthetic promoter, the method comprising: providing a synthetic enhancer described herein or produced by a method described herein; and operably linking the synthetic enhancer to a core promoter (e.g., a core promoter described herein).

In some embodiments, a synthetic enhancer described herein may comprise a nucleic acid fragment or variant of any one of SEQ ID NOs: 2 to 54695 having promoter enhancing activity. In some embodiments, a synthetic enhancer described herein may comprise a nucleic acid fragment encompassing at least 50, 55, 60, 65, 70, 75, 80, 85, 90, 95, or 100 contiguous nucleotides of any one of SEQ ID NOs: 2 to 54695. In some embodiments, a synthetic enhancer described herein may comprise a nucleic acid fragment encompassing at least two adjacently concatenated highly palindromic subsequences of any one of SEQ ID NOs: 2 to 54695. A nucleic acid sequence at least 50, 55, 60, 65, 70, 75, 80, 85, 90, 95, or 100% identical overall, or over a segment of at least 50, 55, 60, 65, 70, 75, 80, 85, 90, 95, or 100 contiguous nucleotides, with respect to any one of SEQ ID NOs: 2 to 54695. In some embodiments, a synthetic enhancer described herein may comprise a nucleic acid sequence that hybridizes under stringent conditions to the full complement of any one of SEQ ID NOs: 2 to 54695. Polynucleotides comprising such sequences are suitable for example as probes, primers, and/or molecular tools for identifying, validating, or discovering novel synthetic enhancers and/or transcription-modulating binding sites. In some embodiments, the stringent conditions comprise hybridization in 6× sodium chloride/sodium citrate (SSC) at about 45° C. followed by one or more washing steps in 0.2× SSC. 0.1% SDS at about 50° C. to about 65° C. In some embodiments, a synthetic enhancer described herein may comprise a nucleic acid sequence that is derived from the sequence of any one of SEQ ID NOs: 2 to 54695 and differs therefrom by no more than 10, 15, 20, 25, 30, 35, 40, 45, or 50 nucleotides. In some embodiments, a synthetic enhancer described herein may comprise concatenated highly palindromic subsequences upstream of (or 5′-relative to) a gene (or gene name) identified in the Sequence Listing filed herewith with respect to any one of SEQ ID NOS: 2 to 54695.

In some embodiments, the core promoter may comprise or consist of the nucleotide sequence of SEQ ID NO: 1, or a variant or fragment thereof having promoter activity. In some embodiments, the core promoter may comprise or consist of a nucleic acid fragment encompassing at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides of SEQ ID NO: 1. In some embodiments, the core promoter may comprise or consist of a nucleic acid sequence at least 50, 55, 60, 65, 70, 75, 80, 85, 90, 95, or 100% identical overall, or over a segment of at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides, with respect to SEQ ID NO: 1. In some embodiments, the core promoter may comprise or consist of a nucleic acid sequence that hybridizes under stringent conditions to the full complement of SEQ ID NO: 1. In some embodiments, the stringent conditions comprise hybridization in 6× sodium chloride/sodium citrate (SSC) at about 45° C. followed by one or more washing steps in 0.2× SSC, 0.1% SDS at about 50° C. to about 65° C. In some embodiments, the core promoter may comprise or consist of a nucleic acid sequence that is derived from SEQ ID NO: 1 and differs therefrom by no more than 10, 15, 20, or 25 nucleotides.

In some embodiments, described herein is a synthetic promoter suitable for driving transcription of a DNA sequence of interest, wherein the synthetic promoter is as described here, or is constructed by a method as described herein. In some embodiments, the synthetic promoter described herein may comprise or consist of a nucleic acid sequence of any one of SEQ ID NOs: 54696, 54700, 54698, or 54701 to 54710, or a variant or fragment thereof having promoter activity. In some embodiments, the synthetic promoter may comprise or consist of a nucleic acid fragment encompassing at least 50, 55, 60, 65, 70, 75, 80, 85, 90, 95, or 100% identical overall, or over a segment of at least 50, 55, 60, 65, 70, 75, 80, 85, 90, 95, or 100 contiguous nucleotides of any one of SEQ ID NOs: 54696, 54700, 54698, or 54701 to 54710. In some embodiments, the synthetic promoter may comprise or consist of a nucleic acid sequence at least 50, 55, 60, 65, 70, 75, 80, 85, 90, 95, or 100% identical overall, or over a segment of at least 50, 55, 60, 65, 70, 75, 80, 85, 90, 95, or 100 contiguous nucleotides, with respect to any one of SEQ ID NOs: 54696, 54700, 54698, or 54701 to 54710. In some embodiments, the synthetic promoter may comprise or consist of a nucleic acid sequence that hybridizes under stringent conditions to the full complement of any one of SEQ ID NOs: 54696, 54700, 54698, or 54701 to 54710. In some embodiments, the stringent conditions comprise hybridization in 6× sodium chloride/sodium citrate (SSC) at about 45° C. followed by one or more washing steps in 0.2× SSC, 0.1% SDS at about 50° C. to about 65° C. In some embodiments, the synthetic promoter may comprise or consist of a nucleic acid sequence that is derived from any one of SEQ ID NOs: 54696, 54700, 54698, or 54701 to 54710 and differs therefrom by no more than 10, 15, 20, 25, 30, 35, 40, 45, or 50 nucleotides.

In some embodiments, described herein is an expression cassette or expression vector comprising a synthetic enhancer as described herein or produced by a method as described herein, operably linked a core promoter (e.g., a core promoter as described herein). In some embodiments, a synthetic promoter, expression cassette, and/or vector as described herein may be for use in gene therapy. In some embodiments, a synthetic promoter, expression cassette, and/or vector as described herein may be for use in genome editing, for example wherein the synthetic promoter drives expression of an endonuclease (e.g., an RNA-guided endonuclease) and/or a guide RNA.

### Computer-Implemented Applications

In some aspects, described herein is a computer-implemented process for constructing a synthetic enhancer. The process generally comprises: (a) inputting or receiving a nucleotide sequence of a promoter of interest; (b) identifying probable palindromic subsequences in the nucleotide sequence of the promoter of interest; (c) selecting and extracting highly palindromic subsequences from amongst the probable palindromic subsequences, the highly palindromic subsequences being those having a palindromic density above a palindromic density threshold; and (d) concatenating some or all of the extracted highly palindromic subsequences to produce a synthetic enhancer having an overall length that is less than that of a contiguous segment of the promoter of interest that comprises all the concatenated highly palindromic subsequences. In some embodiments, the computer-implemented process is a cloud-based computer-implemented process. In some embodiments, the computer may be configured to implement a method as described herein.

In some aspects, described herein is a non-transitory computer-readable medium storing processor-executable instructions, the instructions when executed by a processor cause the processor to perform a method as described herein, and optionally outputting sequence information to a user.

### Implementations

In some implementations, all probable palindromes of a given promoter sequence of interest are identified, and for each nucleotide in the promoter, the total number of times the nucleotide participates in a probable palindrome is calculated. The summation of probable palindromes may then be graphed to create a palindromic density graph to determine subsequences that are more palindromic (e.g., by setting an enhancement threshold) than would be expected in random sequences. These palindromic subsequences are then extracted and concatenated to form the synthetic enhancer sequence of the promoter of interest. The synthetic promoter may then be assembled by fusing the synthetic enhancer to a core promoter. As described herein, this method was then applied across all the promoters in the Homo sapien hg38 genome and in the Mus musculus version mm 10 genome to create a database of shortened synthetic enhancers. The results shown herein demonstrate that palindromic density of a given sequence in the enhancer region of promoters can be a predictor of the capacity of the sequence to partake in transcription factor binding and thus can be used to design shortened synthetic enhancers that can be concatenated with a core promoter.

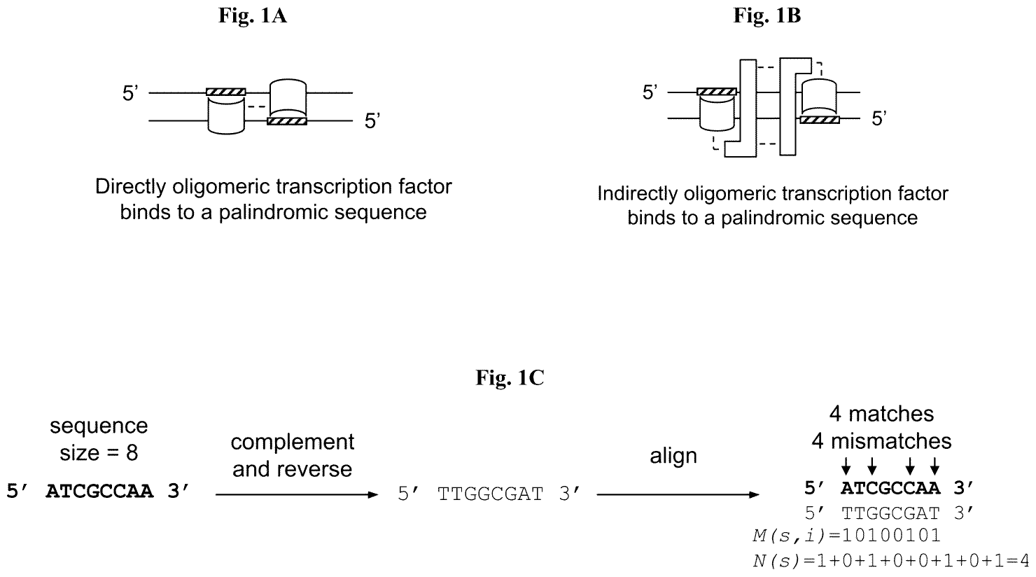

Referring to FIGS. 1A and 1B, transcription factors and most DNA binding proteins are typically associated with oligomers, such as dimers, trimers, and tetramers, thus consistent with the binding sequence being symmetric or palindromic. For example, FIG. 1A shows a direct binding of oligomeric transcription factors to a palindromic sequence. Even with transcription factors that do not have palindromic binding sequences, another binding site in the antisense strand of the promoter could easily create a higher-order palindromic sequence. FIG. 1B shows an indirect binding of oligomeric transcription factors to a palindromic sequence.

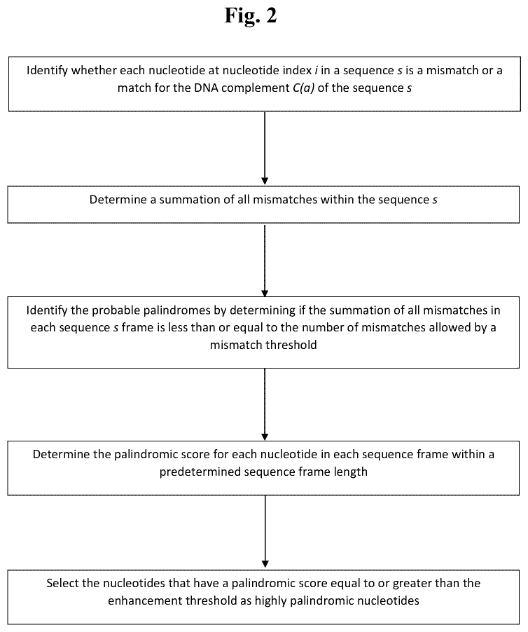

In some embodiments, a palindromic density metric as described herein may be employed to determine the palindromic density of specific subsequences. Referring now to FIG. 2, a mismatch indicator function M(s, i) is used to identify whether each nucleotide at nucleotide index i in a sequence s is a mismatch or a match for the DNA complement C(a) of the sequence s. The mismatch indicator function M(s, i) for each nucleotide in the subsequence s was determined by the equation:

$$M ( s , i ) = \{ \begin{matrix}1 , if {s}_{i} \neq C ( {s}_{L ( s ) - i + 1} ) \\ 0 , otherwise\end{matrix}$$

in which s is the subsequence, i is a nucleotide index, L(s) is the length of the subsequence s and C(s_{L(s)-i+1}) is a DNA complement of the nucleotide, which is identified by s_{L(s)-i+1}. For example, when the nucleotide in the subsequence s at the nucleotide index i is adenine, the DNA complement of the nucleotide (C(s_{L(s)-i+1})) would be thymine. Similarly, when the nucleotide is thymine, guanine, or cytosine, the DNA complement is adenine, cytosine, or guanine, respectively. The mismatch indicator function M(s, i) is 1 if there is a mismatch with the DNA complement C(a) of a specific nucleotide a at nucleotide index i and is 0 when there is a match at index i. For example, as shown in FIG. 1C, in an 8-nucleotide subsequence frame of 5′ ATCGCCAA 3′ has a DNA complement C(a) of 5′ TTGGCGAT 3′, indicating 4 mismatches (bolded). The mismatch indicator function M(s, i) determines these mismatches for each nucleotide index i.

The summation of all mismatches for a sequence s within a promoter of interest can then be determined by the equation:

$$N ( s ) = {\sum }_{i = 1}^{i = L ( s )} M ( s , i ) .$$

In the example shown in FIG. 1C, the summation of all mismatches N(s) is 4.

To determine whether a particular subsequence s is a probable palindrome, a probable palindrome indicator function P(s) is calculated with the following equation:

$$P ( s ) = \{ \begin{matrix}1 , if N ( s ) \leq Cutoff ( L ( s ) ) \\ 0 , otherwise\end{matrix}$$

in which Cutoff(p) is a mismatch threshold.

In some embodiments, the mismatch threshold is the number of mismatches expected from between the top 1% and the top 15% of randomly generated sequences of the same length (i.e., within the 85^{th} to 99^{th} percentile). In some embodiments, the mismatch threshold is the number of mismatches expected from the top 2% to 3% of randomly generated sequences of the same length (i.e., within the 97^{th} to 98^{th} percentile). In an exemplary embodiment discussed herein, a subsequence of a particular length was defined as a probable palindrome if the number of mismatches was less than or equal to the number of mismatches in the top 2% to 3% of randomly generated subsequences of the same length.

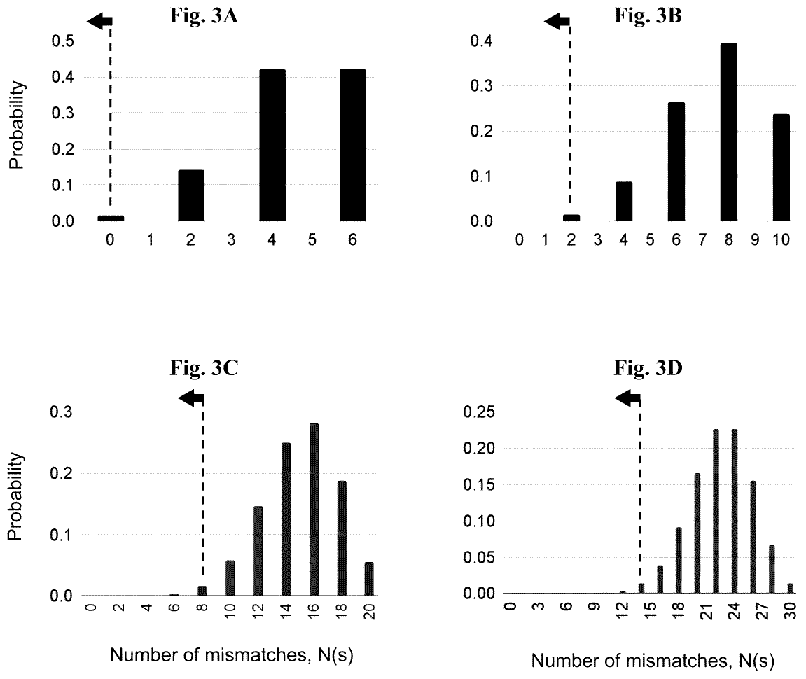

To empirically determine the propensity of palindromic mismatches in subsequences having a length of 6 nucleotides, all unique combinations of 6 nucleotides were generated, yielding 4,096 subsequences. The number of mismatches was calculated for every subsequence and tabulated as a histogram as shown in FIG. 3A. The probability of 0 mismatches was 1.58% and the probability of 0 to 2 mismatches increased to 15.59%; accordingly, 0 mismatches were allowed in probable palindromes having a length of 6 nucleotides (i.e., within the top 2 to 3%). The same procedure was repeated for all sequence having lengths ranging from 6 to 10 nucleotides, as shown in FIG. 3B. For subsequences with lengths of between 11 and 50nucleotides, 1,000,000 randomly generated subsequences were used to create the corresponding histograms to estimate the probability, as shown in FIGS. 3C and 3D. As shown in FIG. 3, the probability of mismatches in the top 2% to 3% (represented by a vertical broken line) of mismatches found in random subsequences of example lengths of 6 (FIG. 3A), 10 (FIG. 3B), 20 (FIG. 3C), and 30 (FIG. 3D) nucleotides was determined to be less than or equal to 0, 2, 8, and 14, respectively. Subsequences of even length, such as the subsequence shown in FIG. 1C, only exhibit even summations of mismatches N(s) and sequences having an odd number of nucleotides have an odd summation of mismatches N(s). The allowed total number of mismatches when using a mismatch threshold within the 97^{th} to 98^{th} percentile for each length of subsequence between 6 and 50 nucleotides is listed in Table 1.

TABLE 1

| Palindrome length | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|
| Allowed mismatches | 0 | 1 | 0 | 1 | 2 | 3 | 4 | 5 | 4 | 5 | 6 | 7 | 6 | 7 | 8 |
| Palindrome length | 21 | 22 | 23 | 24 | 25 | 26 | 27 | 28 | 29 | 30 | 31 | 32 | 33 | 34 | 35 |
| Allowed mismatches | 9 | 10 | 11 | 10 | 11 | 12 | 13 | 14 | 15 | 14 | 15 | 16 | 17 | 18 | 19 |
| Palindrome length | 36 | 37 | 38 | 39 | 40 | 41 | 42 | 43 | 44 | 45 | 46 | 47 | 48 | 49 | 50 |
| Allowed mismatches | 18 | 19 | 20 | 21 | 22 | 23 | 22 | 23 | 24 | 25 | 24 | 25 | 26 | 27 | 28 |

It is to be understood that the mismatch thresholds listed in Table 1 is the number of mismatches expected from the top 2% to 3% of randomly generated subsequences of the same length (97^{th} to 98^{th} percentile); however, the mismatch threshold can vary and can include a mismatch threshold that allows more or less mismatches in a given enhancer sequence length.

Once the probable palindrome indicator function P(s) is calculated, the palindromic score S(s, i) of each nucleotide in the promoter sequence s can be determined with the following equation:

$$S ( s , i ) = {\sum }_{p = y}^{p = x} {\sum }_{w = max ( i - p + 1 , 1 )}^{w = min ( i , L ( s ) - p + 1 )} P ( {s}_{w … ( w + p - 1 )} )$$

wherein p is the length of subsequence frame, which has a maximum nucleotide length of x and a minimum nucleotide length of y. The palindromic score S(s, i) represents the number of probable palindromes of different lengths and subsequence frames the nucleotide participates in.

The subsequence window length p used to determine probable palindromes can vary depending on factors, such as the number of nucleotides in known transcription factor binding sequences in the particular species and/or promoter. In an exemplary embodiment, the length of subsequences used to determine probable palindromes was set between 6 and 50 nucleotides (x=50, y=6), corresponding to known transcription factor binding sequences that are as short as 6 nucleotides, such as the DNA-binding domain of Engrailed (EngHD) binding 5′ TAATTA 3′, and transcription factor binding sequences that are as long as 50 nucleotides, such as the DNA-binding domain of Listeria phage A118 integrase. The palindromic content of transcription factor binding sequences that are longer than 50 nucleotides are still captured by considering all the subsequences within a 6 to 50 nucleotide frame. However, it is to be understood that a subsequence frame of more or less than 6 to 50 nucleotides can be used.

Referring now to FIGS. 4A to 4D, palindromic density graphs showing the tabulated palindromic scores S(s, i) of each nucleotide in the promoter sequence are shown. Many transcription factors bind relatively short and degenerate palindromes, such as the serum response factor binding the consensus 5′ CCWWWWWWGG 3′ (SEQ ID NO: 54711), which can often occur by chance. Accordingly, stronger reasons are needed to consider a short palindrome as being a highly palindromic subsequence, such as the probability of the sequence being involved in the transcriptional activity. As described herein, the density of palindromes in a subsequence is a useful measure in determining that significance.

The palindromic density graphs shown in FIGS. 4A and 4C were determined by evaluating all subsequences between 6 to 50 nucleotides of a promoter of interest to determine if each nucleotide met the criterion for being a probable palindrome (i.e., calculating the probable palindrome indicator function P(s) for each nucleotide). Each nucleotide in the original sequence received a tally of 1 for every probable palindrome the nucleotide was involved with (FIGS. 4A and 4C). These tallies comprised an individual nucleotide's palindromic score S(s, i), with the theoretical limit (i.e. all evaluated subsequences being probable palindromes) equal to 1260.

As shown in FIGS. 4A to 4D, long palindromes were observable as sharp peaks (FIGS. 4A and 4B), whereas overlapping palindromes were observable as flatter peaks (FIGS. 4C and 4D). The protein structures of transcription factors binding to DNA in the protein databank were shown to typically bind around 6 to 10 nucleotides on the DNA. Accordingly, longer palindromic sequences are theorized as a collection of transcription factors that are each recognizing their individual targets that together form a longer palindromic sequence, as opposed to a single transcription factor. For example, transcription factors such as TBX5 and CTCF are homodimers, where each monomer binds non-adjacent DNA sites creating DNA looping.

To evaluate the overall sequence, the average palindromic score A(s) for a given sequence was determined. The average palindromic score A(s) is equal to the average of palindromic scores of each nucleotide in the given sequence and can be determined by the following equation:

$$A ( s ) = \frac{1}{L ( s )} \cdot {\sum }_{i - 1}^{i - L ( s )} S ( s , i )$$

As shown herein, the palindromic density graphs of real promoters have a higher average palindromic score A(s) than that of randomly generated sequences.

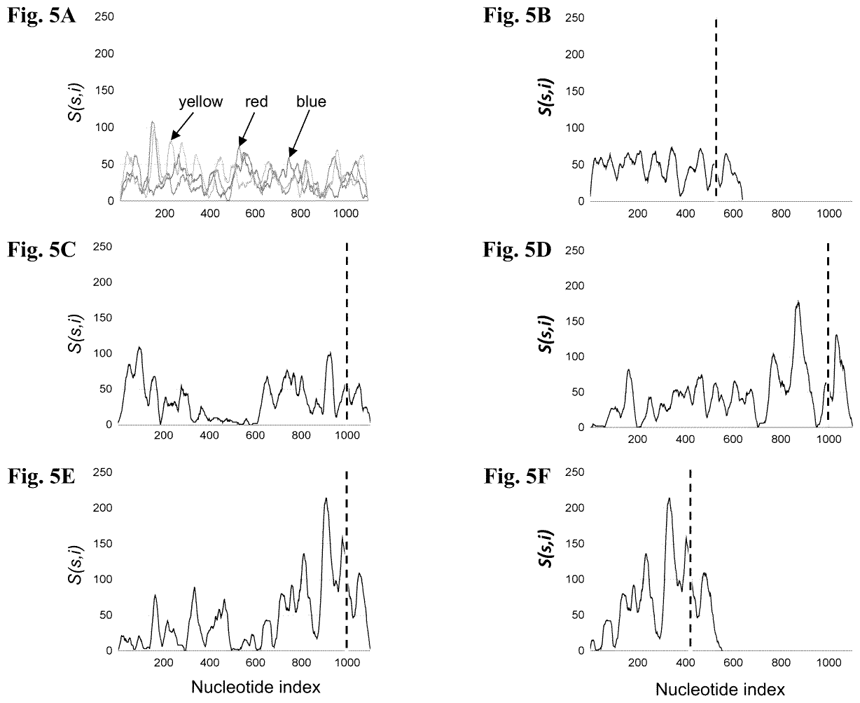

Referring now to FIG. 5A, a palindromic density line graph represented by the palindromic score S(s, i) at each nucleotide index i is shown for three random sequences marked as yellow, red, and blue. To determine the expected palindromic score of a random nucleotide, palindromic density graphs were created for 1,000,000 random sequences having a length of 1101 nucleotides to mimic the analysis of human and mouse promoter sequences. It is understood that any size of random sequences may be generated for the purpose of creating the palindromic density graphs. In an exemplary embodiment, a sequence size of 1101 nucleotides was chosen to mimic the size of larger human and mouse promoters. As shown in FIG. 5A. the palindromic density graphs of random sequences typically had peaks randomly distributed throughout the sequences. The average palindromic score A(s) of these random sequences was 30.55, with the maximum S(s, i) recorded as 264 (Table 2). It should be noted that the first and the last 49 nucleotides in each sequence generally have lower palindromic scores because equation S(s, i) computes the palindromic score using fewer possible sequence frames. When considering the maximum experimental length of 50 nucleotides for probable palindromes, a nucleotide would have a full palindromic score only if there are at least 49 nucleotides upstream and downstream, which is not the case for the end nucleotides. Thus, only the center 1003 nucleotides (excluding 49 nucleotides on each end) would have full tallies (hereafter, named fully scored nucleotides). To calculate the expected palindromic score of a random nucleotide, 1,003,000,000 fully scored nucleotides were averaged, resulting in an average palindromic score of fully scored nucleotides A^{FS}(s) of 31.55 (Table 2).

Referring now to FIGS. 5B to 5F, a palindromic density line graph represented by the palindromic score S(s, i) of each nucleotide at nucleotide index i in a given promoter sequence is shown for a cytomegalovirus immediate-early (CMV) promoter (FIG. 5B), a human insulin promotor (FIG. 5C), a human desmin promoter (FIG. 5D), a human synapsin-1 promoter (FIG. 5E), and a truncated human synapsin-1 promoter (InvivoGen) (FIG. 5F). The nucleotide index of the transcription start site is represented by a dotted vertical green line. Interestingly, the mouse promoter sequences have a cytosine-guanine content (GC-content) of 51.5%, whereas human promoter sequences have a GC-content of 54.5% (Table 6). When random sequences were generated using GC-content consistent with mouse or human sequences, higher GC-content resulted in a slightly higher average palindromic score of fully scored nucleotides A^{FS}(s) of 31.74 and 33.07. respectively (Table 7) (p <0.00000000001, Wilcoxon rank sum test, two-sided, n=1,000,000).

TABLE 2

| Criterion | Random sequences | Mouse promoters | Human promoters |
|---|---|---|---|
| Number of sequences | 1000000 | 25111 | 29598 |
| Sequence length | 1101 | 1101 | 1101 |
| Number of degenerate sequences | 0 | 12 | 1 |
| discarded | | | |
| Average A(s) | 30.55 | 41.99 | 47.97 |
| Average A^{FS}(s) (fully scored | 31.55 | 43.15 | 49.39 |
| nucleotides) | | | |
| Maximum S(s, i) | 281 | 806 | 822 |
| Minimum S(s, i) | 0 | 0 | 0 |

| | Number | % | Number | % | Number | % |
|---|---|---|---|---|---|---|
| Number of sequences with A(s) > | 498948 | 49.89% | 21039 | 83.82% | 26422 | 89.27% |
| 30.55 (i.e. the average A(s) of | | | | | | |
| random sequences) | | | | | | |

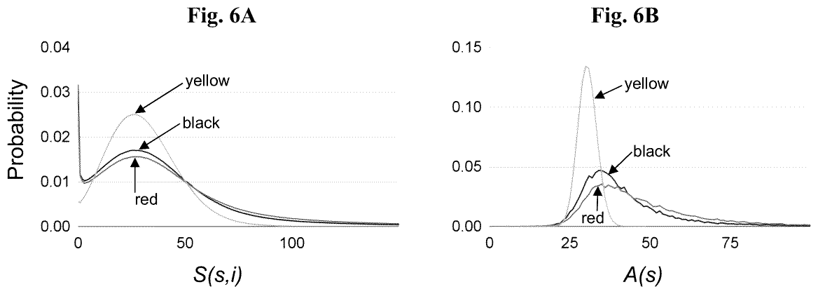

Referring now to FIG. 6A, a probability distribution of the palindromic score S(s, i) of each nucleotide in a given subsequence is shown for random sequences (yellow), mouse genome promoters (black), and human genome promoters (red). FIG. 6B shows the probability distribution of the average palindromic score A(s) for random sequences (yellow), mouse genome promoters (black), and human genome promoters (red). The analysis of the human genome promoters (red) was performed on version hg38 of the human genome and the mouse genome promoters (black) was performed on version mm10 of the mouse genome. The human and mouse sequences analyzed were of the same length as the random sequences (yellow), excluding degenerate sequences. The palindromic score S(s, i) and the average palindromic score of fully scored nucleotides A^{FS}(s) was determined for each nucleotide in the given sequences.

The evaluated human hg38 sequences and mouse version mm10 sequences comprised 1101 nucleotide sequences encompassing 1000 nucleotides upstream of the TSS and 100 nucleotides downstream of the TSS, as determined from Dreos et al., 2017. However, it should be noted that any number of nucleotides upstream and downstream of the TSS can be analyzed. In some embodiments, the size of the sequence can be up to 5000 nucleotides in length. In some embodiments, the sequence of the promoter of interest can comprise from about 400 to about 5000 nucleotides upstream of the TSS to 0 to about 200 nucleotides downstream of the TSS.

When the promoters from the hg38 (Homo sapien) and version mm 10 (Mus musculus) genomes were compared against the randomly generated sequences of the same length, the genome promoters were more palindromic than randomly generated sequences. The average palindromic score of fully scored nucleotides A^{FS}(s) was 41.99 for the version mm 10 mouse promoters and 47.97 for the hg38 human genome promoters, as shown in Table 2 (p<0.00000000001. Wilcoxon rank sum test, two-sided, n=25,099).

Although the number of random sequences was around 33 and 40 times higher than the number of evaluated human and mouse promoters, respectively, the maximum palindromic score S(s, i) in human genome promoters (822) and in mouse genome promoters (806) was around 65% of the theoretical limit and 3 times higher than that of the random sequences (281), which reached only 22.48% of the theoretical limit. Human and mouse promoters were generally more palindromic than the random sequences as 89% of the human promoter sequences and 84% of the mouse promoter sequences had a higher average palindromic score A(s) than the average palindromic score A(s) of the random sequences (30.55). The maximum average palindromic score A(s) for human and mouse genome promoters were 411.48 and 199.87, respectively, both more than four times larger than the maximum average palindromic score A(s) of random sequences, 49.03 (Table 3). Interestingly, human and mouse promoters also had sequences with an average palindromic score A(s) as low as 1.19, much below the corresponding minimum score of random sequences, suggesting that the lack of palindromes may be associated with their functionality. These sequences were usually dominated by non-pairing nucleotides, such as Cytosine-Thymine (CT) rich sequences yielding low palindromic scores. The existence of these abnormally non-palindromic sequences explains the spike in nucleotides with very low palindromic scores (<5), as shown in FIG. 6A.

TABLE 3

| | | Palindromic | Mouse Gene ID | Palindromic | Human Gene ID | Palindromic |
|---|---|---|---|---|---|---|
| | | Score | (Enhancer | Score | (Enhancer | Score |
| | | S(S, i) | SEQ ID NO:) | S(S, i) | SEQ ID NO:) | S(S, i) |
| Top 5 | 1 | 281 | Spryd4_1 (44407) | 806 | HIPK2_1 (11790) | 822 |
| sequences | 2 | 249 | Catip_1 (30081) | 794 | TBXAS1_2 (11789) | 822 |
| with the | 3 | 242 | Esm1_1 (48286) | 769 | AFF2_1 (29424) | 814 |
| highest | 4 | 242 | Dach1_1 (49167) | 764 | AFF2_2 (29423) | 814 |
| S(s, i) | 5 | 240 | Dach1_2 (49165) | 764 | CARM1_2 (25019) | 814 |
| Top 5 | 1 | 49.03 | Usp7_2 (50318) | 199.87 | ACTRT3_3 (6284) | 411.48 |
| sequences | 2 | 46.29 | Zcchc14_1 (41865) | 198.46 | ACTRT3_1 (6283) | 344.91 |
| with the | 3 | 46.14 | Tmem117_1 (50029) | 192.37 | API5_1 (15799) | 256.98 |
| highest A(s) | 4 | 45.6 | Ydjc_1 (50411) | 192.35 | SKI_1 (70) | 255.67 |
| | 5 | 45.51 | 4921531C22Rik_1 (33061) | 190.79 | ZNF430_1 (25395) | 249.74 |
| Top 5 | 1 | 18.18 | Gm732_1 (*) | 1.19 | SMIM24_2 (24749) | 5.30 |
| sequences | 2 | 18.68 | Cldn34c4_1 (54388) | 5.73 | FAM167A_4 (12062) | 6.35 |
| with the | 3 | 18.9 | Fcer2a_1 (40797) | 10.51 | CRCP_3 (11092) | 7.27 |
| lowest A(s) | 4 | 18.92 | Mrln_1 (40797) | 11.96 | GCNA_2 (28991) | 8.23 |
| | 5 | 18.93 | Cabp2_1 (52925) | 12.9 | GCNT3_1 (20590) | 9.93 |

* Promoter sequence selected in this analysis (cut-off of 1000 nucleotides upstream of the TSS) gave a palindromic score too low to extract any subsequences.

Referring back to FIG. 5B, an analogous analysis was also performed on the CMV promoter, which is one of the most commonly used constitutive promoter in the literature. The CMV promoter had an average palindromic score A(s) of 45.28, which is also higher than the average palindromic score A(s) of random sequences (30.55) as shown in Table 4. Furthermore, the shape of the CMV promoter palindromic density graph shown in FIG. 5B suggests that the CMV promoter has probable overlapping palindromes consistently distributed throughout the sequence.

Referring now to FIGS. 5C to 5E, the analysis was also completed on promoters used to target expression in particular tissues: human insulin promoter for pancreatic β cells (FIG. 5C); human desmin promoter for muscle cells (FIG. 5D); and human synapsin-1 promoter for neurons (FIG. 5E). These human promoters had high average palindromic scores A(s), as shown in Table 4. The palindromic density graphs for the human promoters also exhibited sharp peaks, suggesting the presence of long, isolated palindromes.

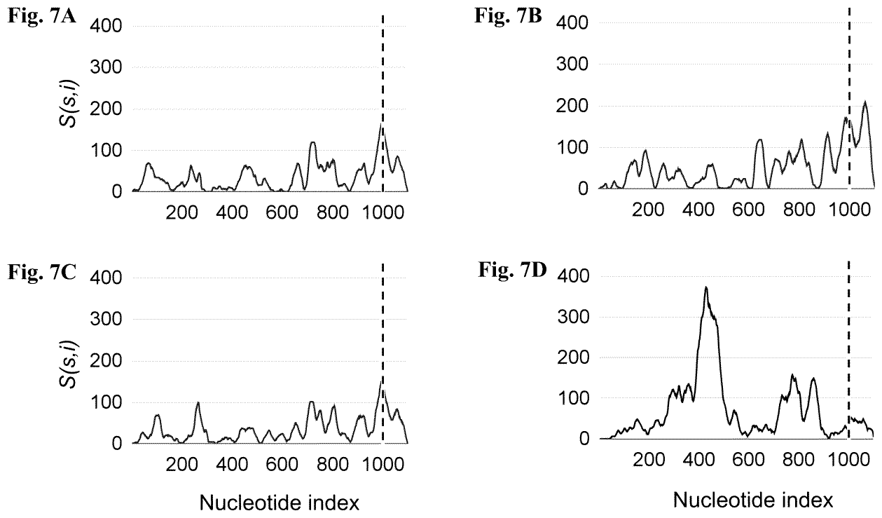

Referring now to FIG. 7, palindromic density line graphs were created for closely related orthologous promoters of the human synapsin-1 promoter in a mouse (FIG. 7A), pig (FIG. 7B), and rat (FIG. 7C), as well as a distantly related orthologous promoters of the synapsin-1 promoter in a fly (FIG. 7D). The closely related orthologous promoters (i.e., mouse, pig, and rat synapsin-1 promoters) had more similarly aligned peaks, which differ from the palindromic density peaks for the distantly related promoter of the fly. Interestingly, when the palindromic density of a truncated human synapsin-1 promoter from InvivoGen was calculated, as shown in Table 4, the truncated human synapsin-1 promoter had a greater average palindromic score A(s) (69.97) than the human synapsin-1 promoter (47.44), suggesting a higher number of palindromes in the upstream region that is proximal to the TSS than the upstream region that is distal to the TSS.

TABLE 4

| Evaluated sequence | A(s) |
|---|---|
| Random | 30.55 |
| CMV promoter | 45.28 |
| Human synapsin-1 promoter (truncated) | 69.97 |
| Human synapsin-1 promoter | 47.44 |
| Mouse synapsin-1 promoter | 36.02 |
| Rat synapsin-1 promoter | 35.15 |
| Pig synapsin-1 promoter | 50.82 |
| Fly synapsin-1 promoter | 71.71 |
| Mouse insulin promoter | 39.68 |
| Mouse desmin promoter | 36.20 |
| Human desmin promoter | 44.77 |
| Human insulin promoter | 34.72 |

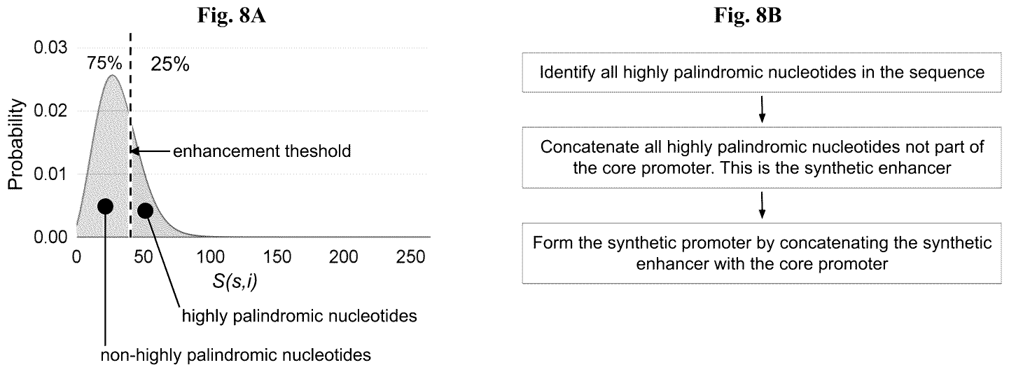

By determining the palindromic scores S(s, i) of each nucleotide within a given promoter sequence, an enhancer sequence can be designed by concatenating the highly palindromic nucleotides in palindromic subsequences found upstream of the TSS. Referring now to FIG. 8A, the distribution of palindromic scores S(s, i) of fully scored nucleotides in random sequences were plotted to determine a threshold for determining what constitutes a highly palindromic nucleotide. As noted above, only the fully scored nucleotides were calculated to avoid scores that are biased to be lower by being on either end of the sequence (i.e., being within a fewer number of sequence frames). The distribution of the palindromic scores S(s, i) in the random sequences had an average palindromic score A^{FS}(s) of 31.55 and followed an extreme value distribution. The distribution of the palindromic scores S(s, i) for the random sequences was then used to determine an enhancement threshold that defines a highly palindromic nucleotide.

In an exemplary embodiment, a highly palindromic nucleotide was defined as a nucleotide within an enhancement threshold that was determined by the top 25% of predetermined palindromic scores of randomly generated sequences (i.e., a highly palindromic nucleotide is a nucleotide within the promoter sequence that has a palindromic score S(s, i) within the 75^{th} percentile of the predetermined palindromic scores S(s, i) of random sequences). In the present example, this definition corresponded to a palindromic score P(s, i) of at least 40. However, it is to be understood that a different enhancement threshold can be used to define a highly palindromic nucleotide. For example, the enhancement threshold can be more tolerant of mismatches, thus corresponding to a larger percentage threshold, such as 40% (i.e. within the 60^{th} percentile), and a lower palindromic score P(s, i) requirement. Alternatively, the enhancement threshold can be less tolerant of mismatches, thus corresponding to a smaller percentage threshold, such as 5% (i.e., within the 95^{th} percentile), and a high palindromic score P(s, i) requirement.

Thus, to design the synthetic enhancer sequence, all of the nucleotides in the promoter sequence that were deemed to be highly palindromic nucleotides were concatenated to produce a synthetic enhancer. In some embodiments, some or all of the nucleotides downstream of the transcription start site, as well as a predetermined number of nucleotides upstream, such as between 50 and 1 nucleotides, can be excluded as these nucleotides typically encompass the core promoter. In an exemplary embodiment, all of the nucleotides downstream of the TSS and 20 nucleotides upstream of the TSS were excluded.

In some embodiments, the highly palindromic nucleotides in the promoter sequence that are adjacent to each other can be considered highly palindromic subsequences. The highly palindromic subsequences can be directly concatenated together to produce the synthetic enhancer. Alternatively, the highly palindromic subsequences can be concatenated via one or more linkers (e.g., 1 to 25 nucleotides in length) interspaced between two or more highly palindromic subsequences. In some embodiments, the linker can comprise a palindromic subsequence or a non-palindromic subsequence. In some embodiments, the linker can comprise a functional sequence, such as a restriction site or a landing site (e.g., integrase, recombinase, or transposase landing site).

Using the method described herein, synthetic enhancer regions for every promoter in the Homo sapien (hg38) and Mus musculus (version mm 10) genome were created. In some embodiments, the Homo sapien synthetic enhancer comprises a nucleotide sequence individually selected from the group consisting of SEQ ID NOs: 2 to 29597. In another embodiment, the Mus musculus synthetic enhancer comprises a nucleotide sequence individually selected from the group consisting of SEQ ID NOs: 29598 to 54695. To create a synthetic promoter using these enhancer sequences, a synthetic enhancer comprising a nucleotide sequence individually selected from the group consisting of SEQ ID NOs: 2 to 54695 can be operably fused, optionally with a linker, to a core promoter. In some embodiments, the core promoter may be a minimal sequence of approximately 50 to 100 nucleotides that enables accurate initiation of transcription at the transcription start site (TSS). However, it should be noted that in some embodiments, the minimal core promoter can encompass a larger or smaller number of nucleotides.

While core promoters appear to be relatively interchangeable, the core promoter from the Cytomegalovirus immediate-early promoter (CMVp, SEQ ID NO: 54700) was used as an exemplary embodiment as CMVp is commonly used in the scientific literature. The minimal CMV core promoter (SEQ ID NO: 1) contains a TATA box and mammalian initiator sequence. Alone, the basal expression of genes controlled by minimal core promoters is significantly lower (and often undetectable) than full-length promoters, which rely on enhancer elements to promote higher levels of expression. This enhancer region contains sequences of transcription factor binding sites that allow for specific expression depending on the transcription factors expressed by the cell. By using the palindromic density of subsequences in the promoter as a metric for the ability of the subsequence to promote similar levels of expression and/or a similar expression profile (i.e., as a predictor for the ability of the subsequence to act as a transcription factor), synthetic enhancers were designed for each promoter of interest to be concatenated with a core promoter, such as the minimal CMV promoter. However, the skilled artisan would understand that any core promoter that is configured to initiate transcription can be used, such as a core promoter from an/a SV40, UbC, EFIA, PGK, or CAGG promoter.

### Experimental Verification

### Example 1

The CMV promoter was used as a test case for synthetic promoter design because the CMV promotor is the most commonly used promoter in the scientific literature to constitutively express genes of interest. The transcription factor binding sites of AP-1 (5′ TGASTCA 3′) and CREB (5′ TGACG 3″) are frequently found in strong constitutive promoters and the CMV promoter has 1 AP-1 site and 11 CREB sites. A synthetic CMV enhancer was created by concatenating highly palindromic subsequences that were identified using the method described herein. Using the method described herein, the sequence of the CMV promoter was reduced from 508 nucleotides to 373 nucleotides, as shown in FIGS. 8C and 8D. Interestingly, the AP-1 site and all the CREB sites of the CMV promoter were conserved. In FIG. 8C, the AP-1 site is boxed, the CREB sites in the forward direction are highlighted in black, and the CREB sites in the reverse direction are underlined.

To create the synthetic CMV promoter (PCMVp, SEQ ID NO: 54696) using the identified highly palindromic subsequences, the 373 nucleotide synthetic enhancer comprising the concatenated subsequences that were identified as being highly palindromic subsequences (SEQ ID NO: 54697) was concatenated to the minimal CMV core promoter (SEQ ID NO: 1).

### Example 2

The mouse synapsin-1 promoter (mSyn1p) was used as a Mus musculus test case. Previous experiments showed that the neuronal-specific expression was abolished when the neuron-restrictive silencer element/repressor element-1 (NRSE/RE-1) was removed from the promoter. The synthetic mSyn1p enhancer was created by concatenating highly palindromic subsequences identified using the method described here. The synthetic mSyn1p enhancer reduced the enhancer of the mSyn1p from 980 nucleotides (as defined in the Eukaryotic Promoter Database of Dreos et al., 2017) to 324 nucleotides, as shown in FIGS. 8E and 8F. Interestingly, the NRSE/RE-1 site known to be required for neuronal specific expression was conserved, as shown highlighted in black in FIG. 8F. To create a synthetic mouse synapsin-1 promoter (PmSyn1p, SEQ ID NO: 54698) using the identified highly palindromic subsequences, the 324 nucleotide synthetic enhancer comprising the concatenated subsequences that were identified as being highly palindromic subsequences was concatenated to a minimal CMV core promoter (SEQ ID NO: 1). The nucleotide sequence of the synthetic enhancer sequence comprises the synthetic enhancer sequence as shown in SEQ ID NO: 53836.

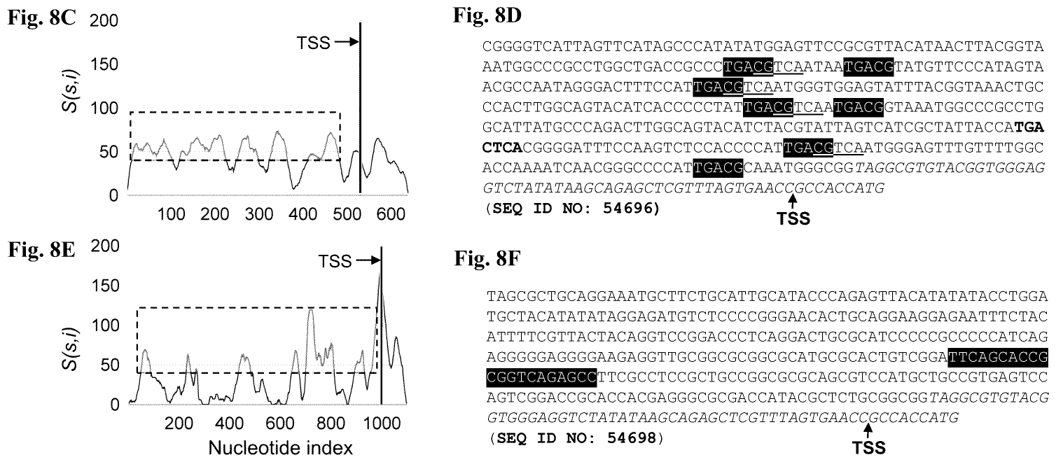

Referring now to FIG. 9, experimental verification of synthetic promoters PCMVp (SEQ ID NO: 54696) and PmSyn1p (SEQ ID NO: 54698) was conducted in living cells. As shown in FIG. 9A, vectors for transiently expressing Venus yellow fluorescent protein under the regulation of PCMVp or PmSyn1p and having a SV40 polyA transcriptional termination sequence (pA) were synthesized. The Venus yellow fluorescent protein was localized to the plasma membrane (pm Venus) with the 12 N-terminal amino acids from Lyn kinase (¹MGCIKSKGKDSA¹²; SEQ ID NO: 54712). All synthesis and subcloning of plasmids was achieved by GenScript™ following a subcloning methodology.

FIG. 9B shows a representation of the translocation of the fluorescent reporter to the plasma membrane by post-translational lipid modification. As the methionine is removed and glycine is lipid-modified for membrane anchoring upon expression, the localization of the Venus yellow fluorescent protein to the plasma membrane indicated that transcription was precisely activated by the upstream promoter.

The pm Venus vectors regulated by PCMBp and PmSyn1p were transfected in HEK293 cells and N2A cells, respectively. The cells were maintained in Dulbecco's Modified Eagle's Medium (DMEM) containing 25 mM D-glucose, 1 mM sodium pyruvate and 4 mM L-glutamine (Invitrogen111) supplemented with 10% Fetal Bovine Serum (FBS) (Sigma-Aldrich) in T25 flasks and incubated at 37° C. and 5% CO₂. Specifically, cells at 90% confluency were transfected with 100 ng of plasmid per well of a 96-well plate for 24 hours with lipofectamine 3000 following the manufacturers' protocol (Thermo Fisher Scientific). Venus positive cells were determined by the percentage of cells in the well that had fluorescence visible through the eyepiece of the microscope.

Prior to imaging. HEK293 cells or N2A cells were plated in 96-well glass-bottom plates (MatTek™). Images were taken with the Olympus IX81™ microscope, using a Lambda™ DG4 xenon lamp for the light source, and a QuantEM™ 512SC CCD camera with a 10× objective or 40× objective (Olympus). Excitation (EX) and emission (EM) filter bandpass specifications for Venus yellow fluorescent proteins (EX: 500/24, EM: 524/27) were used (Semrock™). Images were analysed via ImageJ and μManager software. Imaging was conducted with cells washed and maintained in PBS with CaCl₂ (Sigma). FIGS. 9C and 9D show representative fluorescence images of HEK293 cells transfected with pm Venus regulated by PCMVp at 10× magnification and 40× magnification, respectively. The scale bar at 10× magnification is 100 μm and at 40× magnification is 10 μm. The transfection efficiency of the HEK293 cells transfected with pm Venus regulated by PCMVp was at 70±5% with membrane localization.

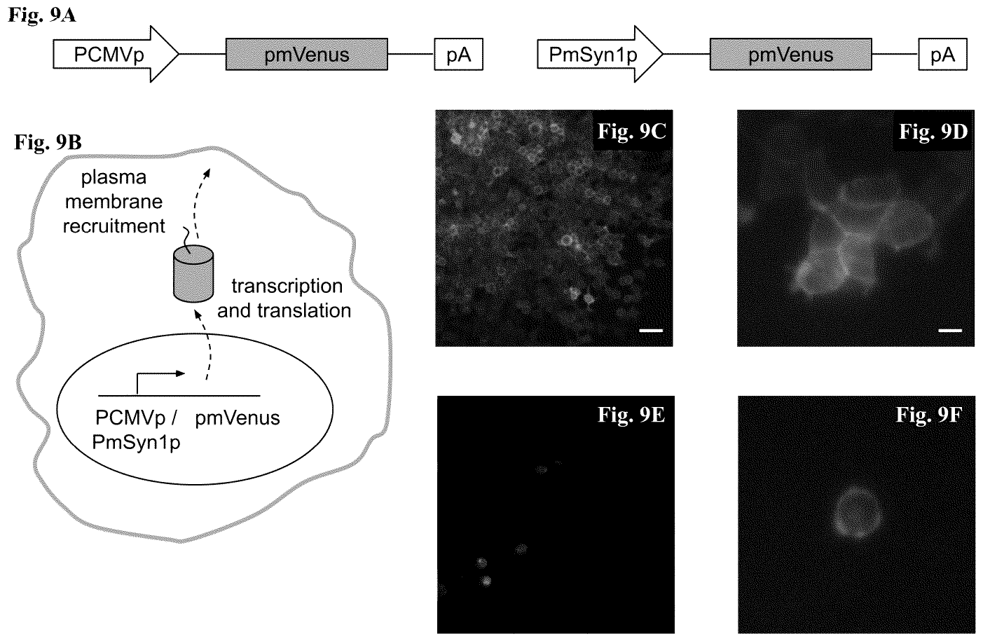

FIGS. 9E and 9F show representative fluorescence images of N2A cells transfected with pm Venus regulated by PmSyn1p at 10× magnification and 40× magnification, respectively. The transfection efficiency of N2A cells transfected with pm Venus regulated by PmSyn1p was 5±3% with membrane localization. The lower transfection efficiency in N2A is due to the less efficient uptake of genetic material, which is a characteristic of this cell line. When the PmSyn1p vector was transfected in HeLa, MDCK, CHO or 3T3 cells, no fluorescence was detected in any experiments (FIG. 9I), despite these cells being able to express fluorescent protein regulated by full length CMVp, as shown in FIG. 10C.

FIGS. 10A to 10C show bar graphs of the normalized fluorescence intensity (f/f₀) of HEK293 cells (FIG. 10A) after transfection with the full length CMVp promoter (SEQ ID NO: 54700) and the synthetic PCMVp promoter (SEQ ID NO: 54696) and N2A cells (FIG. 10B) after transfection with the full length mSyn1p promoter (SEQ ID NO: 54699) and the synthetic PmSyn1p promoter (SEQ ID NO: 54698), where f is the mean fluorescence of regions with cells and f₀ is the mean fluorescence of a similar sized region without cells. The mean normalized fluorescence intensities (f/f₀) were derived from regions encompassing at least 500 cells for HEK293 and 100 cells for N2A. Error bars (s.e.m.) were derived from 3 independent experiments. As can be seen, the normalized fluorescence intensity (f/f₀) for both the full length and synthetic promoters in HEK293 and N2A cells were deemed not significant with an unpaired Student's t test (n.s.). Indeed, as shown in FIGS. 9G and 9H, as well as in FIGS. 10A and 10B, both the PCMVp and PmSyn1p synthetic promoters designed using the method described herein were found to be equally as effective as their full length promoters.

FIG. 10C shows a graph of the percentage of Venus fluorescent in N2A, HeLa, MDCK, CHO and 3T3 cells after transfection with CMVp (full promoter). Error bars (s.d.) were derived from 3 independent experiments with at least 100 cells in the field of view.

### Example 3

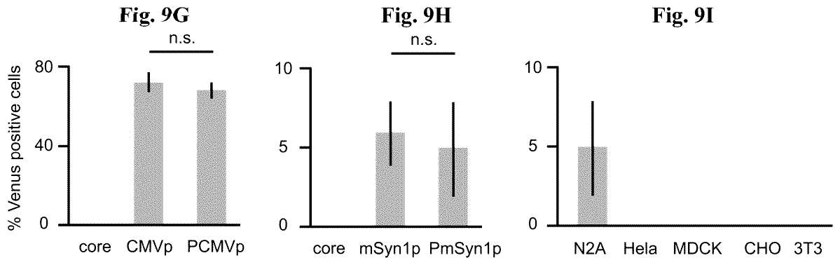

To further verify the broader effectiveness of the method of constructing synthetic promoters, 10 additional shortened human promoters designed using the method described herein were tested in HEK293 cells: CALR, EEF1A1, HSP70, LDHA, NPM1, PKM, RACK1, TUBA1, UBB, and UBC as listed in Table 5. These genes were chosen because the shortened promoters are between 300 to 500 nucleotides and they are among the most well expressed genes in HEK293 cells based on public transcriptome data (GEO ID: GSE165900) on NCBI GEO database. As shown in FIG. 11, all of the tested promoters were active in HEK293 cells with some promoters being as effective as PCMVp.

TABLE 5

| | Promoter | |
|---|---|---|
| Gene | name | Promoter sequence |
| Control | CMV core | TAGGCGTGTACGGTGGGAGGTCTATATAAGCAGAGCTCGTTTAGTGAA |
| | | CCGCCACCATG (SEQ ID NO: 1) |
| SYN1 | mSyn1p | ATGTAGACTAAATATGTGCATGTGGAGGAGGCTGAAAACACATCAGAG |
| | | CTAGCGCTGCAGGAAATGCTTCTGCATTGCATACCCAGAGTTTCCTTG |
| | | CTCATCTGGGAGTCTGTGTTTTTCCTAGATGTGTGCACTTGTGTGAGA |
| | | TTCTCTGGGTGTGAGTCAAAGTGTTATCTGAATGTGTAATGTGTGCTC |
| | | AATATGCTCATGTGTGTTACCCTGAGCTTCTGTGTCTACATATATACC |
| | | TGGATGCCTGTGTGTTCTGTGATGTACATATATATTCTGTCTTTCCTT |
| | | CCTTTTCTATTTGTGTTATTCCATGTGTTCTTTCAGATTCTCACCACC |
| | | AAGGGCAAGGATATGTTAACTACCCAAGTGTCCACCTCCGCCTGTCTG |
| | | GTGATGTTTACGCCACCCCCGTGCTCTTTTCTTTGCCCGACAGAGTTG |
| | | TTATAGGAGATGTCTCCCCGGGAACACTGCAGGAAGGAGAATTTCTAC |
| | | ATTTATGTTCCCCTCTGAGTGTGCTTCTATCCCCAAAATGCCTTCAAA |
| | | GGTGAAAATCAACACTGGAAACCCAAGTATCTGGGAAGGGCAAGAGTG |
| | | TGTAAGTGCAAGTTAGCCTAAGGAATAGGAAGAGGTTGGTAAACAGGG |
| | | TAGGATCGTGGGAGGGAGTTTCGTTACTACAGGTCCGGACCCTCAGGA |
| | | CAAGAACCCCACCCCCACTCCCCAAATTGCGCATCCCCCGCCCCCATC |
| | | AGAGGGGGAGGGGAAGAGGTTGCGGCGCGGCGCATGCGCACTGTCGGA |
| | | TTCAGCACCGCGGTCAGAGCCTTCGCCTCCGCTGCCGGCGCGCACCAC |
| | | CACCTCCCCAGCACCAAAGGCTGACTGACGTCACTCACTAGCCCTCCC |
| | | CAAACTCCCCTTCCTCGCCGCCTTGGTCGCGTCCATGCTGCCGTGAGT |
| | | CCAGTCGGACCGCACCACGAGAGGTGCAAGATAGGGAGGTGCGGGCGC |
| | | GACCATACGCTCTGCGGCGGTAGGCGTGTACGGTGGGAGGTCTATATA |
| | | AGCAGAGCTCGTTTAGTGAACCGCCACCATG (SEQ ID NO: |
| | | 54699) |
| CMV genes | CMVp | CGGGGTCATTAGTTCATAGCCCATATATGGAGTTCCGCGTTACATAAC |
| | | TTACGGTAAATGGCCCGCCTGGCTGACCGCCCAACGACCCCCGCCCAT |
| | | TGACGTCAATAATGACGTATGTTCCCATAGTAACGCCAATAGGGACTT |
| | | TCCATTGACGTCAATGGGTGGAGTATTTACGGTAAACTGCCCACTTGG |
| | | CAGTACATCAAGTGTATCATATGCCAAGTACGCCCCCTATTGACGTCA |
| | | ATGACGGTAAATGGCCCGCCTGGCATTATGCCCAGTACATGACCTTAT |
| | | GGGACTTTCCTACTTGGCAGTACATCTACGTATTAGTCATCGCTATTA |
| | | CCATGCTGATGCGGTTTTGGCAGTACATCAATGGGCGTGGATAGCGGT |
| | | TTGACTCACGGGGATTTCCAAGTCTCCACCCCATTGACGTCAATGGGA |
| | | GTTTGTTTTGGCACCAAAATCAACGGGACTTTCCAAAATGTCGTAACA |
| | | ACTCCGCCCCATTGACGCAAATGGGCGGTAGGCGTGTACGGTGGGAGG |
| | | TCTATATAAGCAGAGCTCGTTTAGTGAACCGCCACCATG |
| | | (SEQ ID NO: 54700) |
| CALR | PhCALRp | AAAAGCCAGGCCGTTTACCCCCTTCATGGAGGGTAGGGTAATAACCCT |
| Bolded | | TTAAAAACAGAGATGCCCCGGTCACAGGGCAGAAGGAGGAGAGGGCTG |
| Sequence: | | GCATTCTTCCCACCGGCCCGCGTGACTGTAGCACCGGGGTGCAGCGAA |
| SEQ ID NO: | | GCGAGCTCTCTCCCATCCCAGGCAGGGGTGGGGGAGCAGCAGGAAAGC |
| 25122 | | CTTGCCCAGCCCCTCCACCTAGAGGGAATGGGAGGGAGGGGTCCCGGT |
| | | CCCGCGTAGACAGCTGCGCTCCCGCCGCGCGCCGGGGTTGGGTTCAGG |
| | | TCTGGTCACATGACCTGGCCTGAGGTGCTCGCGGCCCCCACCCCACCA |
| | | GTGGGCGTCCCCCCCACGCGTGGTCGACCATCATTGGTCGGTGGAACC |
| | | CAGCGTTCCGAGTAGGCGTGTACGGTGGGAGGTCTATATAAGCAGAGC |
| | | TCGTTTAGTGAACCGCCACCATG_(SEQ ID NO: 54701) |
| EEF1A1 | PhEF1p | GCCGCGGGCTGAATTACTTCCACGCCCCTGGCTGCAGTACGTGATTCT |
| Bolded | | TGATCCCGAGCGCTGGGGCCGCCGCGTGCGAATCTGGTGGCACCTTCG |
| Sequence: | | CCTAGCCATTTAAAATTTTTGATTGGGGCCGCGGGCGGCGACGGGGCC |
| SEQ ID NO: | | CGTGCGTCCCAGCGCACATGTTCGGCGAGGCGGGGCCTGCGAGCGCGG |
| 9999 | | CCACCGAGAATCGGCAAGCTGGCCGGCCTGCTCTGGTGCCTGGCCTCG |
| | | CGCCGCCGTGTATCGCCCCGCCCTGGGCGGCAAGGCTGGCCCGGTCGG |
| | | CACCAGGGAGCGGAAAGATGGCCGCTTCCCGGCCCTGCTGCAGGGAGC |
| | | TGTCACCCACACAAAGGAAAAGGGCCTTGTCGCTTCATGTGACTCCAC |
| | | GGAGTACCGGGCGCCGTCCAGGCACCTCGACCCACACTGAGTGGGTGG |
| | | TAGGCGTGTACGGTGGGAGGTCTATATAAGCAGAGCTCGTTTAGTGAA |
| | | CCGCCACCATG_(SEQ ID NO: 54702) |
| HSP70 | PhHSP70p | AGACACGACCGCGTCCTGAGGCGGTTCTGCTGCCTCCCGACAGTTGCC |
| Bolded | | GTAGGGAAGGTGCTGGGAGGCCTGCCGAGCTAACCCGCCCCACCCCGC |
| Sequence: | | GGCGGCCTGGCGGCTCCCTCCAATCCCAATCCTGGGGGGCCGTGAGCG |
| SEQ ID NO: | | AGCAGCCCTAGTGGCACCCCCGGCCAAGATCCCGGCTAGCGCCGCTAT |
| 16979 | | CCGCCCCCTCCCTCCCGCGGAAGCTGGGGGCGCATGCGTAGAGGTGGA |
| | | CGCTCCCCTCCCCCGCCCGGGGTAACTGAGGACTCCCGCGCGCGGACT |
| | | CGCTGCGCCCCACCCTCCCTTTCCCCGGGGCCGTCCGGAGAGCGGGGG |
| | | CGAGCTTGAAAGTAGGGGGGGGCCCCTTCTGGTAGGCGTGTACGGTGG |
| | | GAGGTCTATATAAGCAGAGCTCGTTTAGTGAACCGCCACCATG (SEQ |
| | | ID NO: 54703) |
| LDHA | PhLDHAp | GTTGCAGTGAGCCGAGATCAGCCCACTGCACTCCAGTTAATAATTGAG |
| Bolded | | GCTGCAGGAAGCCATGATCACGCCACTGCCCTCCAGCCTGGGTGACAG |
| Sequence: | | AGTGAGACCCTGCTGACAGTTCTTGGAATGTACATTTGACTGCAAGGC |
| SEQ ID NO: | | CTGAGAGGCCAAGGCTTCACTGTGCAGACTGACTGACTGCTAGGCATT |
| 15642 | | TTCTTCCTTTCCAGGTTTCATGGATGAGGCCTGACTCAGGCTCATGGC |
| | | TCCGACCCCGGCTTCTGTGGAGCAGTCTGCCGGTCGGTTGTCTGGCTG |
| | | CGCGCGCCACCCGGGCCTCTCCAGTGCCCCGCCTGGCTCGGCATCCAC |
| | | CCCCAGCCCGACTCACACGTGGGTTCCCGCACGTCCGCCGGCCCCCCC |
| | | CGCTGACGTCAGCATAGGCGTGTACGGTGGGAGGTCTATATAAGCAGA |
| | | GCTCGTTTAGTGAACCGCCACCATG_(SEQ ID NO: 54704) |
| NPM1 | PhNPMlp | TTGTTTGATATGTTGAGGCTTAAAAAAAAAAGATCTTCAGAACGCCCC |
| Bolded | | AATGCCCGCGGGGTGCTGGGGTGCCTCTTCTTTCATCAGAGTCGGCCC |
| Sequence: | | ACCCTCCGAGCTCTTCAGACAGAGCTGAAAAACTCATTCGAGCCGGCT |
| SEQ ID NO: | | AACCGCTAAGGGCTGCCGACGCCATTTTGCAGGGTGGGCTGCGCAGAC |
| 8931 | | TCTTGGCACGCGTGCGCACAGGCGGTACGAGTGCGCGTGCTCGGTGGG |
| | | AGCCCGCGGAGTACGCTTCGGAGCACGCGCGCGGAGGCAAGCGCTCTA |
| | | GGCGTGTACGGTGGGAGGTCTATATAAGCAGAGCTCGTTTAGTGAACC |
| | | GCCACCATG_(SEQ ID NO: 54705) |
| PKM | PhPKMp | TGCTGTAAGAAAAGTTCCAACAATACCAGTCTCCGAGGGTGCGCCAGA |
| Bolded | | GCAGACGGGCCGGGAGAATGCTGCCCCGGAACCCATAAATCTGGGCCC |
| Sequence: | | TGCCCAGGTAGGCCGGGACAGCTGGGGTGGCCTGGGCCGCCCCATCTG |
| SEQ ID NO: | | GCAGCCCAACTTGGCGGCAACAGGTGGCCCGGCGCCCGGGGGTCTGGG |
| 20744 | | AGGAAAGTCGCTCCGGGGGCGGGCCCCGTTGCCCCGCCGCGTCCCCAT |
| | | TCGAAAGGGCAACCTGCCCGCGCGTTCCGCCGCCGCCGCCGCGCTTCC |
| | | TCCTGAAGGTGACTGCGCCCGCGGGGACGCAGGGGGCGGGGCCCGGGT |
| | | CGCCCGGAGCCGGGATTGGGCAGAGGGCGGGGCGGCGGAGGGATTGCG |
| | | GCGGCCCGCAGCGGGTAGGCGTGTACGGTGGGAGGTCTATATAAGCAG |
| | | AGCTCGTTTAGTGAACCGCCACCATG_(SEQ ID NO: 54706) |
| RACK1 | PhRACK1p | TTTATTATTTTATTTGCTGCCCAGGCTGGAGTGCAGTGGCGCGATCTA |
| Bolded | | CTGCAAAGGGATTCTCCCCGAGTAACTCAGTCCAGCCTGGACTAAGTC |
| Sequence: | | CTTCCGGAGTTGGCACAGTTTTAAAGTTTATTTTTAACATTTTAATAC |
| SEQ ID NO: | | TCTACTTTTTAAATTGCCCATCCACGATGTGGAACAGGCGGAGCTCGA |
| 9090 | | GCTATGCCACATAAAGCCTTTTTAAACACTTGAATGTGCTTGTTTCAG |
| | | AGTGTAGGCGTGTACGGTGGGAGGTCTATATAAGCAGAGCTCGTTTAG |
| | | TGAACCGCCACCATG (SEQ ID NO: 54707) |
| TUBA1 | PhTUBA1bp | TCTGAAATGTGTTTTTAAATCTCCTTTTCAAAAAGCCCTTAGGACTGT |
| Bolded | | TCCATAAAGTTGATGGCTTAGATCTAAAATTGGGGCCTACTTTGCCCT |
| Sequence: | | TTTAACAGCTAATGACTGAAATACTATTTGCTTCCAGCGTTTGGTTCA |
| SEQ ID NO: | | GGTTGCGGGAGGCCGACTCGCGCCCGCCCCTCGGCAGGCCCTCGCAGC |
| 17649 | | CATGCGCCGCACACTGCAGTACTGGCGCGCGTTGCCCGCAGGCGCGGC |
| | | AGACCCCACCCCGCGGCCGCGCGAGGGGAGGGGGCTCGGGGCTCGGAG |
| | | CCCGCCTCTGCGGCGGCCAGGCCGGGCGCGGAGTGGGCGCGCGGGGCC |
| | | GGAGGAGGGGCCAGCGACCGCGGCACCGCCTGTGCCCGCCCGCCCCTC |
| | | CGCAGCCGCTACTTAAGAGGCTAGGCGTGTACGGTGGGAGGTCTATAT |
| | | AAGCAGAGCTCGTTTAGTGAACCGCCACCATG |
| | | (SEQ ID NO: 54708) |
| UBB | PhUBBp | CCTTGGCCAGGCTGGTCTTGACAGGCGTGAGCCTCCGCGCCCGGCCAG |
| Bolded | | GGGCGCGCGTTTTTAATATCGGGTGCCACGCCGTCCCGCTTCTGAGGC |
| Sequence: | | GCGGCGGCCCACTTTGGCAGGCCGAGGCGGGAGATCGCGCCATTGCAC |
| SEQ ID NO: | | TCCAGCTCCCGCCGGAATTCAGGACGGCGCGCCTGTGCGGCGCACGCG |
| 22803 | | CGCTCAGTTACTTAGCAACCTCGGCGCTAAGCCACCCGCGCTGCAAGG |
| | | AAGTTTCCAGAGCTTTCGAGGAAGGTTTCTTCAACTCTCATCTGATAA |
| | | TTTTCTTATATTTTCCTAAAGAAGGAAGAGACTGCCTCTCGGGAGGTT |
| | | GGGCGCGGCGAACTACTTGGGTGATAAGTGACGCAACACTCGTTGCAT |
| | | AAATTTAGGCGTGTACGGTGGGAGGTCTATATAAGCAGAGCTCGTTTA |
| | | GTGAACCGCCACCATG (SEQ ID NO: 54709) |
| UBC | PhUBCp | AACGCGGGGTTCGCGACCCGAGGGGACCGCGGGGGCTGAGGGGAGGGG |
| Bolded | | CCGCGGAGCCGCGGCTAAGGAACGCGGGCCGCCCACCCGCTCCCGGTG |
| Sequence: | | CAGCGGCCTCCGCGCCGGGTTTTGGCGCCTCCCGCGGGCGCCCCCCTC |
| SEQ ID NO: | | CTCACGGCGAGCGCTGCCACGTCAGACGAAGGGCGCAGCGAGCGTCCT |
| 18659 | | GATCCTTCCGCCCGGACGCTCAGGACAGCGGCCCGCTGCTCATAAGAC |
| | | TCGGCCTTAGAACCCCAGTAAAGTAGTCCCTTCTCGGCGATTCTGCGG |
| | | AGGGATCTCCGCCGGGTGTGGCACAGCTAGTTCCGTCGCAGCCGGGGC |
| | | TGCTGGGCTGGCCGGGGCTTTCGTGGCCGCCGGGCCGCTCGGTGGGAC |
| | | GGAGGCTCCCGAGTCTGGCAAGAACCCAAGGTCTTGAGGCCTTCGCTA |
| | | ATGCGGGCAGTGCACCCGTACCTTTGGGAGCGCGCGCCCTCGTTAGGC |
| | | GTGTACGGTGGGAGGTCTATATAAGCAGAGCTCGTTTAGTGAACCGCC |
| | | ACCATG (SEQ ID NO: 54710) |

### Example 4

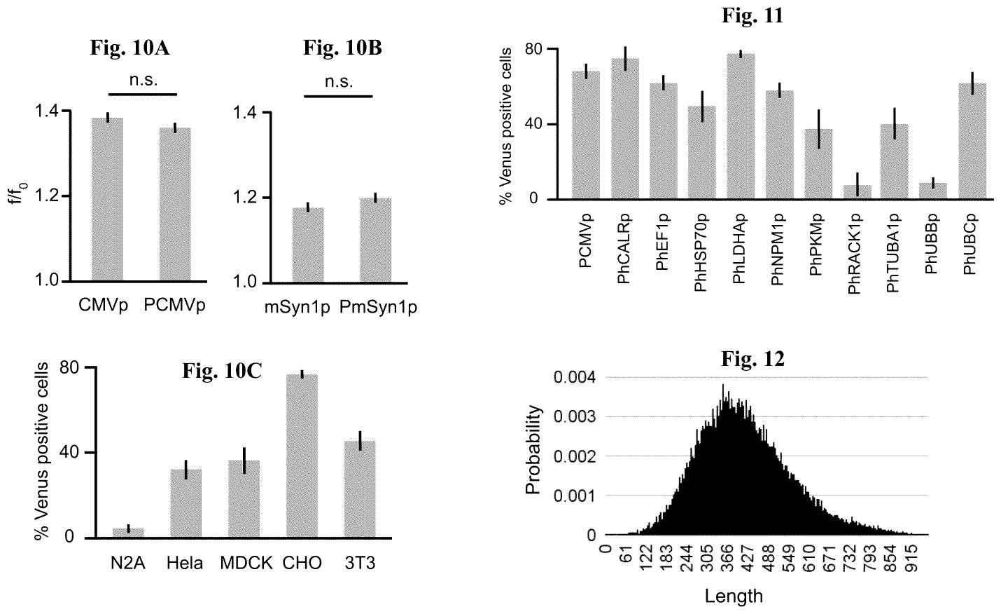

The method of constructing synthetic promoters described herein was applied to all Homo sapien and Mus musculus promoters identified in the hg38 human genome and the version mm 10 mouse genome, respectively. The analysis showed that promoter sequences have a higher palindromic density when compared to randomly generated sequences. As shown in FIG. 12, the average length of the resulting synthetic enhancers was 413 nucleotides and the length of the enhancer sequence increased as the average of palindromic scores A(s) increased.

TABLE 6

| | Mouse | Human |
|---|---|---|
| | promoters | promoters |
| Number of sequences | 25111 | 29598 |
| Sequence length | 1101 | 1101 |
| Number of degenerate sequences | 12 | 1 |
| discarded | | |
| Number of nucleotides evaluated | 27633999 | 32586297 |
| Proportion of adenine (A) | 0.242 | 0.227 |
| Proportion of thymine (T) | 0.242 | 0.228 |
| Proportion of cytosine (C) | 0.256 | 0.271 |
| Proportion of guanine (G) | 0.259 | 0.274 |
| Proportion of GC-content | 0.515 | 0.545 |

TABLE 7

| | Balanced GC- | Mouse GC- | Human GC- |
|---|---|---|---|
| | content | content | content |
| Number of sequences | 1000000 | 1000000 | 1000000 |
| Sequence length | 1101 | 1101 | 1101 |
| Average A(s) | 30.55 | 30.74 | 32.03 |
| Average A^{FS}(s) | 31.55 | 31.74 | 33.07 |
| (fully scored nucleotides) | | | |
| Maximum S(s, i) | 281 | 309 | 386 |
| Minimum S(s, i) | 0 | 0 | 0 |

Altogether, these results show a synthetic enhancer region can be designed for a promoter of interest using palindromic density as a metric for determining highly palindromic subsequences.

### REFERENCES

Dreos et al., “The Eukaryotic Promoter Database in Its 30th Year: Focus on Non-Vertebrate Organisms”. Nucleic Acids Res 2017, 45 (D1), D51-D55.

1. A method of constructing a synthetic enhancer, the method comprising:
identifying probable palindromic subsequences in a promoter of interest;
selecting and extracting highly palindromic subsequences from amongst the probable palindromic subsequences, the highly palindromic subsequences being those having a palindromic density above a palindromic density threshold; and
concatenating some or all of the extracted highly palindromic subsequences to produce a synthetic enhancer having an overall length that is less than that of a contiguous segment of the promoter of interest that comprises all the concatenated highly palindromic subsequences.

2. The method of claim 1, wherein:
(a) identifying the probable palindromic subsequences comprises:
defining a candidate subsequence of a predetermined length in the promoter of interest;
generating a complement or reverse complement of the candidate subsequence;
comparing the candidate subsequence with a DNA complement or reverse complement to identify the number of mismatches; and
identifying the candidate subsequence as the probable palindromic subsequence if the number of mismatches is the same or lower than a mismatch threshold corresponding to the number of mismatches expected from comparable randomly generated sequences; and/or
(b) selecting the highly palindromic subsequences based on the palindromic density comprises determining a palindromic nucleotide score S(s, i) for each individual nucleotide in the probable palindromic subsequence, the palindromic nucleotide score correlating with a number of probable palindromic subsequences of different lengths and different subsequence frames in which the nucleotide participates, and optionally plotting a palindromic density graph of palindromic nucleotide score as a function of nucleotide position within the promoter of interest.

3. The method of claim 2, wherein:
(a) the candidate subsequence's length is set at a minimal length of at least 4, 5, 6, 7, 8, 9, or 10 nucleotides, and/or a maximal length of up to 30, 35, 40, 45, 50, 55, 60, 65, 70, 75, 80, 85, 90, 95, 100, 125, or 150 nucleotides;
(b) the candidate subsequence is compared with its reverse complement by performing a sequence alignment to identify the number of mismatches;
(c) the mismatch threshold corresponds to the number of mismatches expected from the most palindromic randomly generated sequences of the same length as the candidate subsequence, such as the number of mismatches expected within a 60^{th}, 65^{th}, 70^{th}, 75^{th}, 80^{th}, 85^{th}, 90^{th}, 95^{th}, 96^{th}, 97^{th}, 98^{th}, or 99^{th} percentile of randomly generated sequences of a same length; or
(d) any combination of (a) to (c).

4. The method of claim 2, wherein
(a) comparing the number of mismatches is determined by mismatch indicator function M (s, i):
$$M ( s , i ) = \{ \begin{matrix}1 , if {s}_{i} \neq C ( {s}_{L ( s ) - i + 1} ) \\ 0 , otherwise\end{matrix} ;$$
where s is a candidate subsequence of the promoter of interest, i is a nucleotide index, L(s) is a length of the subsequence s, and C(s_{L(s)-i+1}) is the DNA complement of nucleotide s_{L(s)-i+1};
(b) selecting the highly palindromic subsequences based on the palindromic density further comprises determining an overall palindromic density sequence score for each of the probable palindromic subsequence, the overall palindromic density sequence score correlating with the palindromic nucleotide scores for all or substantially all individual nucleotides in the probable palindromic subsequence;
(c) the palindromic nucleotide score S(s, i) is determined by:
$$S ( s , i ) = {\sum }_{p = y}^{p = x} {\sum }_{w = max ( i - p + 1 , 1 )}^{w = min ( i , L ( s ) - p + 1 )} P ( {s}_{w … ( w + p - 1 )} )$$
wherein p is a palindrome length of each probable palindromic subsequence, and the palindrome length has a maximum number of nucleotides equal to x, and a minimum number of nucleotides equal to y; or
(d) any combination of (a) to (c).

5. The method of claim 4, wherein comparing the number of mismatches further comprises performing a summation of the mismatches N(s):
$$N ( s ) = {\sum }_{i = 1}^{i = L ( s )} M ( s , i ) .$$

6. The method of claim 5, wherein probable palindromic subsequences are determined by calculating a probable palindrome indicator function P(s):
$$P ( s ) = \{ \begin{matrix}1 , if N ( s ) \leq Cutoff ( L ( s ) ) \\ 0 , otherwise\end{matrix} ;$$
where Cutoff(p) is a mismatch threshold corresponding to the number of allowed mismatches for a sequence of length p.

7. (canceled)

8. (canceled)

9. The method of claim 1, wherein;
(a) the palindromic density threshold is based on the expected palindromic densities of comparable randomly generated sequences;
(b) the extracted highly palindromic subsequences are concatenated with one or more intervening synthetic linker sequences therebetween, wherein at least one of the one or more intervening synthetic linker sequences comprises a palindromic subsequence, a non-palindromic subsequence, or binding site (e.g., a restriction site or a landing site, such as an integrase, recombinase, or transposase landing site);
(c) the extracted highly palindromic subsequences are concatenated without intervening synthetic linker sequences therebetween;
(d) the promoter of interest comprises a promoter from a mammalian genome; or
(e) the method further comprises synthesizing a polynucleotide comprising the synthetic enhancer.

10. The method of claim 9, wherein the palindromic density threshold is within a 60^{th}, 65^{th}, 70^{th}, 75^{th}, 80^{th}, 85^{th}, 90^{th}, or 95^{th} percentile of the expected palindromic densities of comparable randomly generated sequences.

11. (canceled)

12. The method of claim 4, wherein:
(a) x is 30, 35, 40, 45, 50, 55, 60, 65, 70, 75, 80, 85, 90, 95, 100, 125, or 150 nucleotides;
(b) y is 4, 5, 6, 7, 8, 9, or 10 nucleotides;
(c) wherein the length of the sequence (L(s)) of the promoter of interest is less than 1 000 000, 500 000, 250 000, 200 000, 150 000, 100 000, 50 000, 25 000, 20 000, 15 000, 10 000, 7500, 5000, 4000, 3000, 2000, 1500, or 1101 nucleotides;
(d) the overall palindromic density sequence score is calculated based on the average of the palindromic nucleotide scores of all individual nucleotides in the probable palindromic subsequence according to the function:
$$A ( s ) = \frac{1}{L ( s )} \cdot {\sum }_{i = 1}^{i = L ( s )} S ( s , i )$$
where i is the nucleotide index; or
(e) any combination of (a) to (d).

13. (canceled)

14. (canceled)

15. (canceled)

16. The method of claim 1, wherein the promoter of interest:
(a) has a length of less than 1 000 000, 500 000, 250 000, 200 000, 150 000, 100 000, 50 000, 25 000, 20 000, 15 000, 10 000, 7500, 5000, 4000, 3000, 2000, 1500, 1250, or 1000 nucleotides;
(b) comprises between 200 and 5000 nucleotides upstream of a transcription start site of the promoter of interest;
(c) comprises 0 to 200, 0 to 150, 0 to 100, or 20 to 100 nucleotides downstream of the transcription start site of the promoter of interest;
(d) comprises less than 1000 nucleotides upstream of the transcription start site of the promoter of interest; or
(e) any combination of (a) to (d).

17. (canceled)

18. The method of claim 9, wherein the mammalian genome is a Homo sapien genome (e.g., hg38) or a Mus musculus genome (e.g., mm10).

19. (canceled)

20. The method of claim 9, wherein the synthetic enhancer is fused to a core promoter, or to a core promoter operably fused to a polynucleotide sequence to be transcribed.

21. The method of claim 20, wherein the synthetic enhancer is heterologous with respect to the core promoter and/or with respect to the polynucleotide sequence to be transcribed and/or wherein the core promoter sequence is a minimal CMV promoter.

22. (canceled)

23. The method of claim 1, further comprising: providing the synthetic enhancer produced by the method of claim 1; and operably linking the synthetic enhancer to a core promoter or to a core promoter operably fused to a polynucleotide sequence to be transcribed.

24. The method of claim 1, wherein the synthetic enhancer comprises:
(a) a nucleic acid fragment or variant of any one of SEQ ID NOs: 2 to 54695 having promoter enhancing activity;
(b) a nucleic acid fragment encompassing at least 50, 55, 60, 65, 70, 75, 80, 85, 90, 95, or 100 contiguous nucleotides of any one of SEQ ID NOs: 2 to 54695;
(c) a nucleic acid fragment encompassing at least two adjacently concatenated highly palindromic subsequences of any one of SEQ ID NOs: 2 to 54695;
(d) a nucleic acid sequence at least 50, 55, 60, 65, 70, 75, 80, 85, 90, 95, or 100% identical overall, or over a segment of at least 50, 55, 60, 65, 70, 75, 80, 85, 90, 95, or 100 contiguous nucleotides, with respect to any one of SEQ ID NOs: 2 to 54695;
(e) a nucleic acid sequence that hybridizes under stringent conditions to the full complement of any one of SEQ ID NOs: 2 to 54695, optionally wherein the stringent conditions comprise hybridization in 6× sodium chloride/sodium citrate (SSC) at about 45° C. followed by one or more washing steps in 0.2× SSC, 0.1% SDS at 50° C. to 65° C.;
(f) a nucleic acid sequence that is derived from the sequence of any one of SEQ ID NOs: 2 to 54695 and differs therefrom by no more than 10, 15, 20, 25, 30, 35, 40, 45, or 50 nucleotides; or
(g) any combination of (a) to (f).

25. A synthetic promoter suitable for driving transcription of a DNA sequence of interest, wherein the synthetic promoter is as defined in claim 23, or is constructed by the method of claim 24.

26. (canceled)

27. The synthetic promoter of claim 25, for use in gene therapy.

28. The synthetic promoter of claim 25, for use in genome editing, wherein the synthetic promoter drives expression of an endonuclease (e.g., an RNA-guided endonuclease) and/or a guide RNA.

29. A computer-implemented process for constructing a synthetic enhancer, the process comprising:
(a) inputting or receiving a nucleotide sequence of a promoter of interest;
(b) identifying probable palindromic subsequences in the nucleotide sequence of the promoter of interest;
(c) selecting and extracting highly palindromic subsequences from amongst the probable palindromic subsequences, the highly palindromic subsequences being those having a palindromic density above a palindromic density threshold; and
(d) concatenating some or all of the extracted highly palindromic subsequences to produce a synthetic enhancer having an overall length that is less than that of a contiguous segment of the promoter of interest that comprises all the concatenated highly palindromic subsequences.

30. (canceled)

31. The computer-implemented process of claim 29, wherein said computer is configured to implement the method as defined in claim 1.

32. (canceled)
